(19) United States
(12) Patent Application Publication
(10) Pub. No.: US 2021/0241178 A1
(43) Pub. Date: Aug. 5, 2021

(54) COMPUTATIONALLY DERIVED CYTOLOGICAL IMAGE MARKERS FOR PREDICTING RISK OF RELAPSE IN ACUTE MYELOID LEUKEMIA PATIENTS FOLLOWING BONE MARROW TRANSPLANTATION IMAGES

(71) Applicant: Case Western Reserve University, Cleveland, OH (US)

(72) Inventors: Anant Madabhushi, Shaker Heights, OH (US); Sara Arab Yarmohammadi, Shaker Heights, OH (US); Zelin Zhang, Cleveland, OH (US); Patrick Leo, Honeoye Falls, NY (US); Leland Metheny, Shaker Heights, OH (US); Howard Meyerson, Strongsville, OH (US)

(21) Appl. No.: 17/167,438

(22) Filed: Feb. 4, 2021

Related U.S. Application Data
(60) Provisional application No. 62/969,721, filed on Feb. 4, 2020.

(51) Int. Cl. G06N 20/10; G06N 3/00; G06N 3/02; G01N 33/483

(52) U.S. Cl. CPC G06N 20/10; G06N 3/002; G01N 2800/54; G01N 33/4833; G06N 3/02

(57) ABSTRACT

Embodiments discussed herein facilitate determination of risk of relapse of AML post-transplant. One example embodiment is a method, comprising: accessing a digital whole slide image (WSI) comprising a post-transplant bone marrow aspirate from a patient that has acute myeloid leukemia (AML); segmenting one or more myeloblasts on the digital WSI; extracting one or more features from the segmented one or more myeloblasts; providing the one or more features extracted from the segmented one or more myeloblasts to a trained machine learning model; and receiving, from the trained machine learning model, an indication of a risk of relapse of the AML.

### CROSS REFERENCE TO RELATED APPLICATIONS

This application claims the benefit of U.S. Provisional Patent Application No. 62/969,721 filed Feb. 4, 2020, entitled “COMPUTATIONALLY DERIVED CYTOLOGICAL IMAGE MARKERS FOR PREDICTING RISK OF RELAPSE IN ACUTE MYELOID LEUKEMIA PATIENTS FOLLOWING BONE MARROW TRANSPLANTATION IMAGES”, the contents of which are herein incorporated by reference in their entirety.

### BACKGROUND

Acute myeloid leukemia (AML) is a severe hematologic malignancy that disturbs the hematopoietic stem cells of the bone marrow. In AML, stem cell myeloblasts, commonly referred to as ‘blasts,’ do not undergo typical lineage-specific white blood cell (WBC) differentiation. Consequently, these immature WBC's outnumber other normal WBCs and reduce space for healthy WBCs, red blood cells, and platelets in the blood and bone marrow. As a result, AML patients may experience infection, anemia, and poor blood clotting. Thus, detecting myeloblasts and evaluating their quantity play a significant role in diagnosing AML.

Allogenic hematopoietic stem cell transplantation (HCT) is the best post-remission consolidation therapy, frequently the only curative option for patients with adverse AML but is associated with significant co-morbidities and mortality due to graft-versus-host disease and immunosuppression. Additionally, 40%-80% of AML patients relapse following HCT. Relapse rates can be reduced through myelosuppressive chemotherapy, but that can cause many side effects due to the suppression of bone marrow activity resulting in reduced production of blood cells. Additionally, relapse patients generally have poor prognosis, since many, especially early relapse cases, can either not endure or are refractory to low-dose or intensive myelosuppressive chemotherapy. Moreover, only a minority of patients can be salvaged in the long term despite therapies such as donor lymphocyte infusions or a second HCT in selected patients. The low response rate, poor improvement under salvage therapy, and substantial side effects of these treatments make it vital to direct them only to high-risk patients.

Since blast quantity is closely related to AML severity, the current gold standard for early detection of relapse post-transplant is pathologic review of aspirates from bone marrow biopsy to evaluate blast proliferation. In this process, a hematopathologist counts approximately 200 cells from randomly chosen regions of a bone marrow aspirate specimen and if 5% or more of the cells are blasts, the patient is considered to have relapsed. The examination of only a small proportion of cells, intra- and inter-observer variation, and the limits of human perception contribute to limit the accuracy and reproducibility of this approach.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates a flow diagram of an example method/set of operations that can be performed by one or more processors to employ a machine learning model to determine a risk of relapse post-transplant for an AML patient, in connection with various aspects discussed herein.

FIG. 2 illustrates a flow diagram of an example method/set of operations that can be performed by one or more processors to construct a machine learning model that can determine a risk of relapse post-transplant for an AML patient, in connection with various aspects discussed herein.

FIG. 3 illustrates a diagram showing an overall workflow and pipeline of the first example study, in connection with various aspects discussed herein.

FIG. 4 illustrates a flowchart outlining the distribution of study cytology slides by cohort of origin and experiment, in connection with various aspects discussed herein.

FIG. 5 illustrates example images showing segmentation results, in connection with various aspects discussed herein.

FIG. 6 illustrates the distribution of myeloblast counting in the two groups of AML patients post-HCT, in connection with various aspects discussed herein.

FIG. 7 illustrates box plots showing the distribution of each feature in the prognostic signature for high-risk and low-risk patients in the training cohort and the validation cohort, in connection with various aspects discussed herein.

FIG. 8 illustrates example images showing the two top LASSO features visualized between relapse and no-relapse patients, in connection with various aspects discussed herein.

FIG. 9 illustrates Kaplan—Meier survival curves for the test set and validation set, in connection with various aspects discussed herein.

FIG. 10 illustrates a flowchart showing the image analysis steps for identifying blasts and then using the shape and appearance and arrangement of the blasts on aspirate cytological images to predict response to HCT, in connection with various aspects discussed herein.

FIG. 11 illustrates example images showing the blast segmentation results, in connection with various aspects discussed herein.

FIG. 12 illustrates violin plots of the four top discriminating features in predicting relapse across the entire dataset of n=39 images, in connection with various aspects discussed herein.

FIG. 13 illustrates violin plots of the four top fractal features in predicting relapse across the entire dataset of n=39 slides, in connection with various aspects discussed herein.

FIG. 14 illustrates images and graphs showing differences in fractal feature expression between patients who will relapse from those who will not, in connection with various aspects discussed herein.

FIG. 15 illustrates example images that visualize the differences of intensity entropy values on blasts in relapse and no-relapse patients, in connection with various aspects discussed herein.

FIG. 16 illustrates a diagram showing the AMLcGAN training process, in connection with various aspects discussed herein.

FIG. 17 illustrates example images and graphs showing the influence of the discriminative feature distribution of the discriminator output on the output of the generator during the training phase, in connection with various aspects discussed herein.

FIG. 18 illustrates example images showing discriminator feature visualization, in connection with various aspects discussed herein.

FIG. 19 illustrates a diagram showing the detailed module structures of AMLcGAN, in connection with various aspects discussed herein.

FIG. 20 illustrates two graphs showing the average adversarial loss and average feature matching loss of the generator and discriminator in each epoch during the AMLcGAN training process, and the segmentation accuracy of the generator on the validation set after each training epoch, in connection with various aspects discussed herein.

FIG. 21 illustrates an example image (left) and magnified region (right) showing visualization of segmentation results, in connection with various aspects discussed herein.

FIG. 22 illustrates example images showing comparison results of different segmentation models, in connection with various aspects discussed herein.

FIG. 23 illustrates a graph showing the evaluation results of AMLcGAN on the test set, in connection with various aspects discussed herein.

FIG. 24 illustrates violin plots demonstrating the difference of blast counts obtained based on AMLcGAN between AML patients groups post-HCT, in connection with various aspects discussed herein.

FIG. 25 illustrates a diagram of an example apparatus that can facilitate constructing, training, and/or employing models that can automatically segment myeloblasts and/or determine a risk of relapse post-transplant for AML based on features of digitized WSI(s) of bone marrow aspirate, in connection with various aspects discussed herein, in connection with various aspects discussed herein.

### DETAILED DESCRIPTION

Various embodiments discussed herein include apparatus, systems, operations, methods, or other embodiments that facilitate constructing, training, and/or employing models that can automatically segment myeloblasts and/or determine a risk of relapse post-transplant for AML based on features of digitized WSI(s) of bone marrow aspirate.

Some portions of the detailed descriptions that follow are presented in terms of algorithms and symbolic representations of operations on data bits within a memory. These algorithmic descriptions and representations are used by those skilled in the art to convey the substance of their work to others. An algorithm, here and generally, is conceived to be a sequence of operations that produce a result. The operations may include physical manipulations of physical quantities. Usually, though not necessarily, the physical quantities take the form of electrical or magnetic signals capable of being stored, transferred, combined, compared, and otherwise manipulated in a logic or circuit, and so on. The physical manipulations create a concrete, tangible, useful, real-world result.

It has proven convenient at times, principally for reasons of common usage, to refer to these signals as bits, values, elements, symbols, characters, terms, numbers, and so on. It should be borne in mind, however, that these and similar terms are to be associated with the appropriate physical quantities and are merely convenient labels applied to these quantities. Unless specifically stated otherwise, it is appreciated that throughout the description, terms including processing, computing, calculating, determining, and so on, refer to actions and processes of a computer system, logic, circuit, processor, or similar electronic device that manipulates and transforms data represented as physical (electronic) quantities.

Example methods and operations may be better appreciated with reference to flow diagrams. While for purposes of simplicity of explanation, the illustrated methodologies are shown and described as a series of blocks, it is to be appreciated that the methodologies are not limited by the order of the blocks, as some blocks can occur in different orders and/or concurrently with other blocks from that shown and described. Moreover, less than all the illustrated blocks may be required to implement an example methodology. Blocks may be combined or separated into multiple components. Furthermore, additional and/or alternative methodologies can employ additional, not illustrated blocks.

Embodiments include apparatus, systems, operations, methods, or other embodiments that can involve constructing, training, and/or employing models that can automatically segment myeloblasts and/or determine a risk of relapse post-transplant for AML based on features of digitized WSI(s) of bone marrow aspirate.

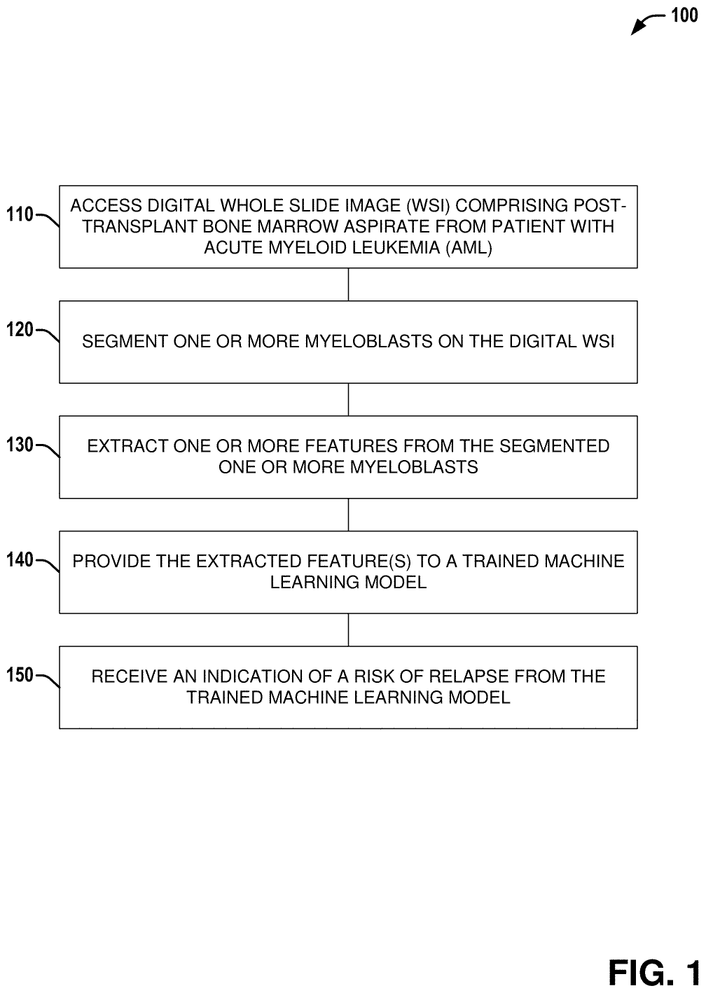

Referring to FIG. 1, illustrated is a flow diagram of an example method/set of operations 100 that can be performed by one or more processors to employ a machine learning model to determine a risk of relapse post-transplant for an AML patient, in connection with various aspects discussed herein. Processor(s) can include any combination of general-purpose processors and dedicated processors (e.g., graphics processors, application processors, etc.). The one or more processors can be coupled with and/or can include memory or storage and can be configured to execute instructions stored in the memory or storage to enable various apparatus, applications, or operating systems to perform the operations. The memory or storage devices may include main memory, disk storage, or any suitable combination thereof. The memory or storage devices can comprise—but is not limited to—any type of volatile or non-volatile memory such as dynamic random access memory (DRAM), static random-access memory (SRAM), erasable programmable read-only memory (EPROM), electrically erasable programmable read-only memory (EEPROM), Flash memory, or solid-state storage.

The set of operations 100 can comprise, at 110, accessing a digital whole slide image (WSI) comprising a post-transplant bone marrow aspirate from a patient that has acute myeloid leukemia (AML). In various embodiments and in the example study discussed below, the digital whole slide image (WSI) can be obtained via a system and/or apparatus implementing the set of operations 100, or can be obtained from a separate medical imaging system (e.g., optical microscope, etc.). Additionally, the digital WSI can be accessed contemporaneously with or at any point prior to performing the set of operations 100.

The set of operations 100 can further comprise, at 120, segmenting one or more myeloblasts on the digital WSI.

The set of operations 100 can further comprise, at 130, extracting one or more features from the segmented one or more myeloblasts.

The set of operations 100 can further comprise, at 140, providing the one or more features extracted from the segmented one or more myeloblasts to a trained machine learning model.

The set of operations 100 can further comprise, at 150, receiving, from the trained machine learning model, an indication of a risk of relapse of the AML.

Additionally, or alternatively, set of operations 100 can comprise one or more other actions discussed herein in connection with determining a risk of relapse post-transplant for an AML patient.

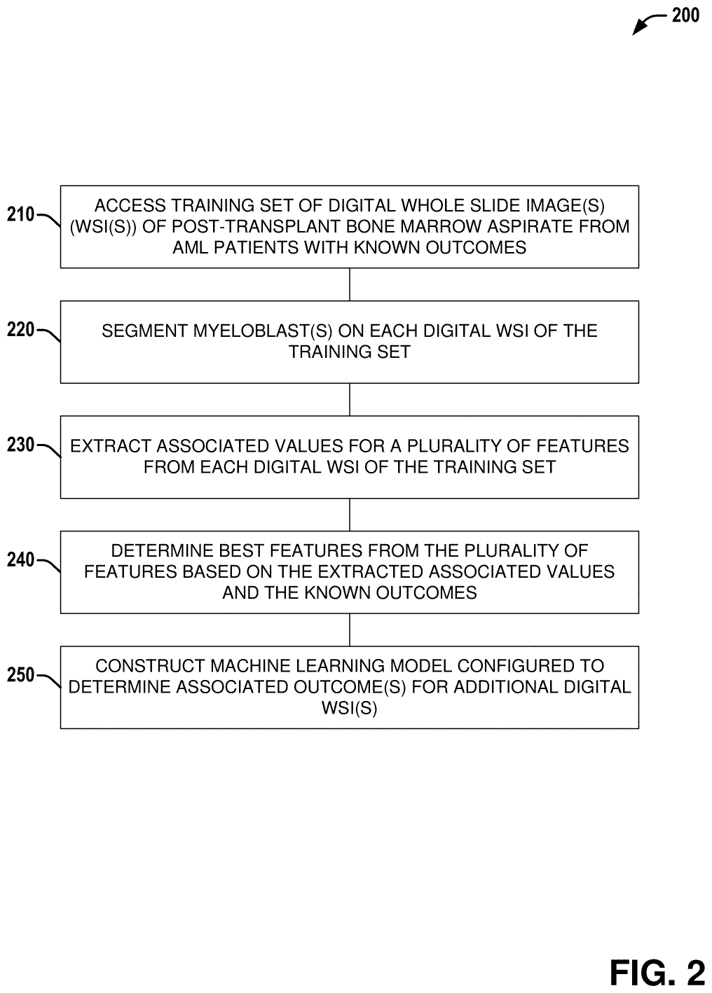

Referring to FIG. 2, illustrated is a flow diagram of an example method/set of operations 200 that can be performed by one or more processors to construct a machine learning model that can determine a risk of relapse post-transplant for an AML patient, in connection with various aspects discussed herein.

The set of operations 200 can comprise, at 210, accessing a training set comprising a plurality of digital whole slide images (WSIs), wherein each digital WSI of the plurality of digital WSIs comprises an associated post-transplant bone marrow aspirate from an associated patient that has acute myeloid leukemia (AML), wherein each digital WSI of the plurality of digital WSIs has a known associated outcome for the associated patient of that digital WSI, wherein the known associated outcome is one of a relapse or a non-relapse. In various embodiments, the digital WSI(s) can be obtained via a system and/or apparatus implementing the set of operations 200, or can be obtained from a separate medical imaging system (e.g., optical microscopy system). Additionally, the digital WSI(s) can be accessed contemporaneously with or at any point prior to performing the set of operations 200.

The set of operations 200 can further comprise, at 220, for each digital WSI of the training set, segmenting one or more associated myeloblasts on that digital WSI.

The set of operations 200 can further comprise, at 230, for each digital WSI of the training set, extracting an associated value for each of a plurality of features from the associated segmented cancer nuclei of that digital WSI.

The set of operations 200 can further comprise, at 240, determining a set of best features from the plurality of features, based at least in part on the known associated outcome for each digital WSI of the training set and on the associated values for each of the plurality of features for each digital WSI of the training set.

The set of operations 200 can further comprise, at 250, constructing a machine learning model configured to determine an additional associated outcome for an additional digital WSI based at least in part on the set of best features.

Additionally, or alternatively, set of operations 200 can comprise one or more other actions discussed herein in connection with constructing a machine learning model that can determine a risk of relapse post-transplant for an AML patient, in connection with various aspects discussed herein.

Additional aspects and embodiments are discussed below in connection with the following example studies.

### Example Study 1: Machine Learning Approach to Predict Risk of Relapse Using Cytological Image Markers in Acute Myeloid Leukemia Patients Post-HCT

The following discussion provides example embodiments in connection with a first example study involving constructing, training, and/or employing models that can automatically segment myeloblasts and/or determine a risk of relapse post-transplant for AML based on features of digitized WSI(s) of bone marrow aspirate.

### Abstract

Allogenic hematopoietic stem cell transplant (HCT) is a curative therapy for acute myeloid leukemia (AML). Relapse post-HCT is the most common cause of treatment failure and is associated with poor prognosis. In the first example study, Wright-Giemsa stained post-HCT aspirate images were collected from 92 AML patients and were randomized into a training set (Sₜ=52) and a validation set (Sᵥ=40). First, a deep learning model was developed to segment myeloblasts. A total of 214 texture and shape descriptors were then extracted from these segmented myeloblasts on cytological images. The pathomic risk-score that was generated by using the least absolute shrinkage and selection operator (LASSO) with a Cox regression model was associated with relapse-free survival on Sᵥ (hazard ratio of 1.57; 95% confidence interval 1.01-2.45). The features identified by LASSO were then used to train a linear discriminant analysis classifier to prognosticate relapse with an area under the receiver operating characteristic curve of 0.75 within Sᵥ.

Significance: Previous studies in machine learning AML diagnosis and prognosis have largely focused on myeloblast detection, segmentation, and counting. The first example study demonstrates the crucial role of myeloblasts chromatin pattern and shape heterogeneity features in prognosticating AML relapse post-HCT and highlight the importance of these features in the development of machine learning prognostic tools.

### Introduction

Acute myeloid leukemia (AML) is a severe hematologic malignancy that disturbs the hematopoietic stem cells of the bone marrow. In AML, stem cell myeloblasts, commonly referred to as ‘blasts,’ do not undergo typical lineage-specific white blood cell (WBC) differentiation. Consequently, these immature WBC's outnumber other normal WBCs and reduce space for healthy WBCs, red blood cells, and platelets in the blood and bone marrow. As a result, AML patients may experience infection, anemia, and poor blood clotting. Thus, detecting myeloblasts and evaluating their quantity play a significant role in diagnosing AML.

Allogenic hematopoietic stem cell transplantation (HCT) is the best post-remission consolidation therapy, frequently the only curative option for patients with adverse AML but is associated with significant co-morbidities and mortality due to graft-versus-host disease and immunosuppression. Additionally, 40%-80% of AML patients relapse following HCT. Relapse rates can be reduced through myelosuppressive chemotherapy, but that can cause many side effects due to the suppression of bone marrow activity resulting in reduced production of blood cells. Additionally, relapse patients generally have poor prognosis, since many, especially early relapse cases, can either not endure or are refractory to low-dose or intensive myelosuppressive chemotherapy. Moreover, only a minority of patients can be salvaged in the long term despite therapies such as donor lymphocyte infusions or a second HCT in selected patients. The low response rate, poor improvement under salvage therapy, and substantial side effects of these treatments make it vital to direct them only to high-risk patients.

Since blast quantity is closely related to AML severity, the current gold standard for early detection of relapse post-transplant is pathologic review of aspirates from bone marrow biopsy to evaluate blast proliferation. In this process, a hematopathologist counts approximately 200 cells from randomly chosen regions of a bone marrow aspirate specimen and if 5% or more of the cells are blasts, the patient is considered to have relapsed. The examination of only a small proportion of cells, intra- and inter-observer variation, and the limits of human perception contribute to limit the accuracy and reproducibility of this approach. Automatic computational image analysis and extraction of sub-visual features could avoid these limitations and yield an objective, accurate prognostic test.

There have been many studies on using computational image analysis for disease prognosis from digitized histologic images of solid tumors. However, there has been less work in the cytopathology space, with the literature largely focused on problems of cell segmentation, WBC classification, and automated cell counting rather than outcome prediction and prognosis, especially in AML. Prior work indicates that fractal dimension (FD) of blast chromatin may be a prognostic factor in acute precursor B lymphoblastic leukemia. The authors found that myeloblast chromatin complexity revealed patterns of DNA methylation and was associated to patient survival. Such literature motivates further investigation of the utility of automated image analysis in leukemia.

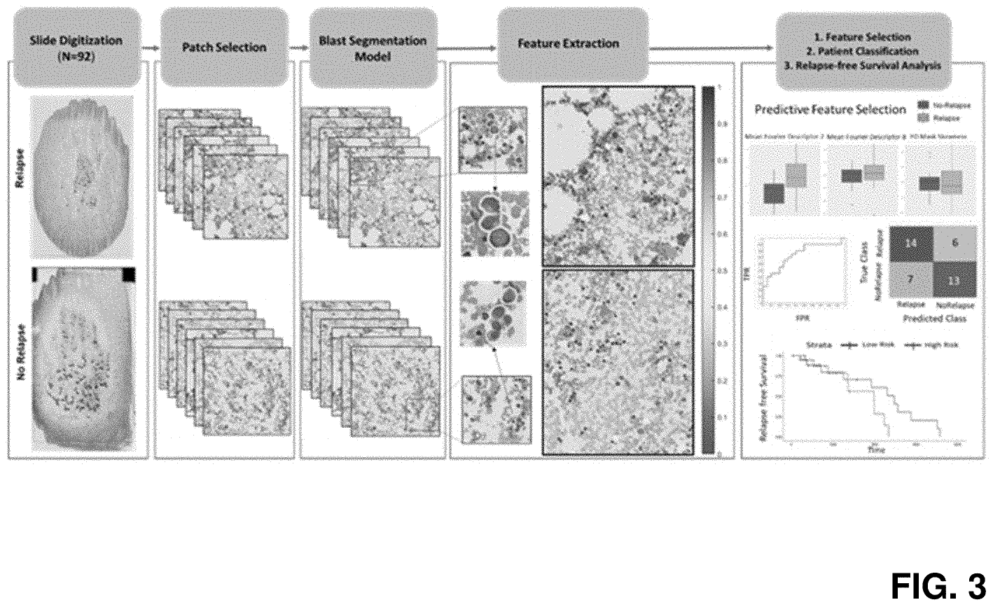

Referring to FIG. 3, illustrated is a diagram showing an overall workflow and pipeline of the first example study, in connection with various aspects discussed herein. First, the whole dataset was divided into training and validation sets. 6 random 512×512 micron tiles were then selected from every Wright-Giemsa stained aspirate slide image. Myeloblasts were segmented on all the tiles and features of blast shape and chromatic pattern were extracted. Features which correlated most with relapse were selected on the training dataset. A classifier was trained using the training cohort, and the performance was validated on the validation set.

The first example study presents a method, as demonstrated in FIG. 3, for post-HCT relapse prognosis using automated analysis of Wright-Giemsa stained bone marrow aspirate images. Acute Myeloid Leukemia cytological image classifier (AMLCIC) uses two quantitative descriptors of blast chromatin appearance selected from 214 features automatically extracted from images. It was found that the machine extracted features could accurately assess patient risk, and these assessments were correlated with clinical and pathologic features.

### Materials and Methods

### Patient Selection

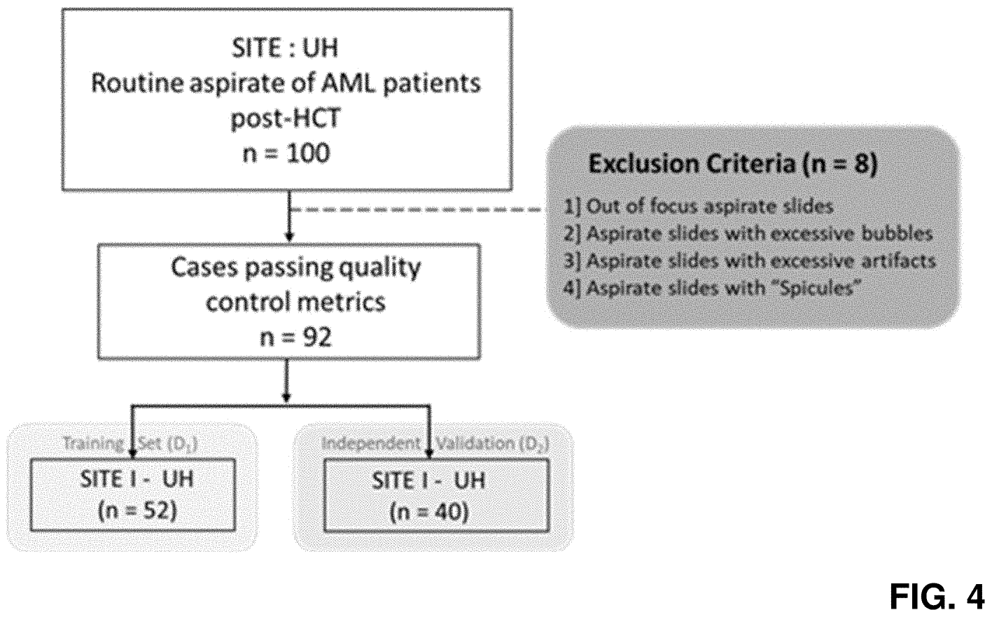

Referring to FIG. 4, illustrated is a flowchart outlining the distribution of study cytology slides by cohort of origin and experiment, in connection with various aspects discussed herein. In an IRB approved protocol, AML patients who underwent HCT at University Hospitals Cleveland Medical Center were reviewed. Bone marrow Wright-Giemsa-stained aspirate slides were collected from n=92 AML patients six to eight weeks after HCT. Of these patients, 48 experienced relapse. All slides were digitized at 40× magnification. Six random non-overlapping 512×512 micron tiles were selected from each digitized aspirate image on regions with dense WBCs and no artifacts, bubbles, etc., for a total of 552 tiles. Patients were divided into training and validation sets, with 40 patients randomly selected to make the validation set (20 of whom had relapse) and the training set containing the rest of 52 patients (28 of whom had relapse) and the testing set containing 40 patients (20 relapsed). Patients who did not experience relapse were censored at the date of the last follow-up.

Relapse-free survival (RFS) was defined as the time interval between the start of treatment (date of HCT) and the date of relapse in AML patients. For censored patients the survival is defined between the HCT date and the last follow up date.

### Image Analysis

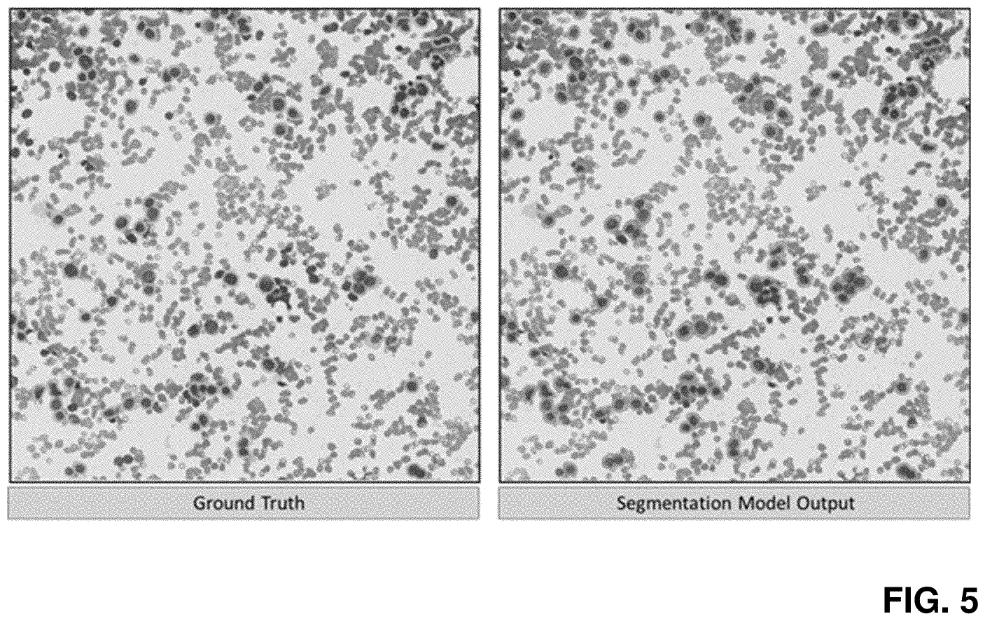

Blast detection and segmentation: A blast segmentation framework based on UNet was trained on 795 64×64 micron patches from 35 patients annotated for myeloblasts by a hematopathologist. Of these, 79 random patches were held out for model testing. On the held out test set, the model yielded a per-pixel true positive rate of 0.99, true negative rate of 0.96, and F1 score of 0.76. Segmentation was then performed on all 552 tiles from n=92 aspirate slide images, and results were visually verified to be suitable for feature extraction. Referring to FIG. 5, illustrated are example images showing segmentation results, in connection with various aspects discussed herein. In FIG. 5, the left image shows the ground truth overlaid on one of the tiles in the training set, and the right image shows the mask resulting from the segmentation model's output overlaid on the same tile.

Feature Extraction: Features pertaining to chromatin patterns, heterogeneity, shape complexity, and shape irregularity were extracted from each segmented myeloblast. Myeloblast shape irregularity also captures the deformation and distortion of the blast boundary. In addition, the fractal properties of chromatin in myeloblasts was measured using FD (fractal dimension) features. The first category of features comprised two features of blast counts and covered area ratio while the second set comprised 52 Haralick texture features. The third category included 64 one dimensional (1 D) and two dimensional (2D) fractal features and 96 shape features to assess morphological and textural changes of blasts within aspirate slides. Haralick texture features were extracted from gray level co-occurrence matrices (GLCMs) to measure heterogeneity on the cell chromatin. FD features can also quantify complexity and irregularity of microscopic anatomic structures and show the fractal nature of chromatin in histologic sections. The mean, median, standard deviation, and skewness of each feature was calculated across all myeloblasts on all tiles from a patient to arrive at a patient-level feature value, and in each feature category of Table 1, below, features are computed using the mean, max, min, and standard deviation of the values across all six tiles. This produced a total of 214 features for each patient.

TABLE 1
Feature categorizations in the first example study

| Feature | Count of Features | Sample |
|---|---|---|
| Category | in Category | Feature |
| Blast Statistics | 2 | Count of blasts |
| | | Area ratio |
| Haralick Texture | 52 | Entropy |
| | | Energy |
| Fractal Dimension | 64 | FD1_Mask |
| | | FD_2D_Image |
| Other Shape Features | 96 | Smoothness |
| | | Perimeter Ratio |

### Statistical Analysis

As number of myeloblasts is the feature that pathologists look for in evaluating these slides, this feature was compared in the relapse and no-relapse groups of the training and validation sets by t-test.

To avoid overfitting due to high complexity of features, a least absolute shrinkage and selection operator (LASSO) was used to identify the most prognostic features from all the features in training set (Sₜ) to build the multivariable signature for prognosticating RFS. The “glmnet” package in R was used for executing the LASSO algorithm. These features were then used in a Cox regression model to produce the pathomic risk score (PRS) for each patient.

The association of the PRS with RFS was first assessed in Sₜ and subsequently validated in validation set (Sᵥ) by using Kaplan-Meier survival analysis. A threshold for the risk score was then identified on Sₜ using the mean of PRS, based on which, patients in both Sₜ and Sᵥ were classified as high-risk or low-risk. Model performance was evaluated by the Kaplan-Meier method, the log-rank test, and Harrell's concordance index (c-index). Multivariable survival analyses with PRS and clinical biomarker was also performed and relative HRs with 95% confidence intervals (CI) were calculated using the Wald test and the G-rho rank test, respectively in R, version 4.0.1.

To find which patients relapse post-HCT, a linear discriminant analysis (LDA) classifier was trained, forming the AML cytological image classifier (AMLCIC) with the set of features identified by LASSO. LDA was used since it was a simple linear classifier with the best performance on the training set. AMLCIC was trained within Sₜ and then evaluated for prognosticating of relapse on Sᵥ. The ability to identify relapse post-HCT was assessed by area under the receiver operating characteristic curve (AUC). Accuracy, sensitivity, and specificity were also computed at the optimal operating point of the ROC curve, the operating point being defined as the threshold that maximized overall accuracy.

### Results

### Patient Characteristics

Among the 48 relapse patients, the median time to relapse was 269 (range: 47 to 1574) days, with 60% of these relapses occurring within 1 year of HCT. Among the 12 patients who relapsed beyond 18 months, the median time to relapse was 2.3 (range, 1.7 to 4.3) years. Table 2, below, summarizes the clinical variables in Sₜ and Sᵥ.

TABLE 2
Summary of clinical variables in training set (Sₜ)
and validation set (Sᵥ)

| Clinical Variable | Sₜ: N (%) | Sᵥ: N (%) |
|---|---|---|
| Age | | |
| >= 50 years | 32(61.5%) | 28(70%) |
| <50 years | 17(32.7%) | 12(30%) |
| unknown | 3(5.8%) | 0 (0%) |
| Gender | | |
| Female | 18(34.6%) | 21(52.5%) |
| Male | 31(59.6%) | 19(47.5%) |
| unknown | 3(5.8%) | 0(0%) |

### Experiment 1: Count of Myeloblasts is not a Significant Biomarker for Discriminating Between Relapse and No-Relapse Patients

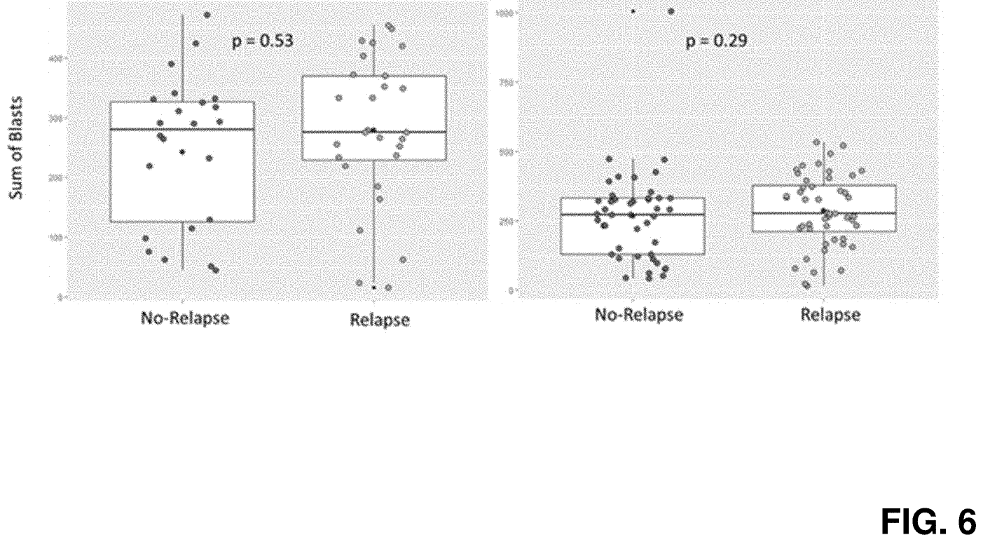

The results show that number of blasts was not significantly different between relapse and no-relapse groups (p>0.05). Referring to FIG. 6, illustrated is the distribution of myeloblast counting in the two groups of AML patients post-HCT, in connection with various aspects discussed herein. FIG. 6 shows a comparison between blast count in the relapse and no-relapse groups, with the left showing the comparison within Sₜ, and the right showing the comparison within Sₜ+Sᵥ.

### Experiment 2: AMLCIC is Prognostic of Relapse Post-HCT

On the independent validation set, AMLCIC was able to distinguish relapse from no-relapse patients with an AUC of 0.71, an accuracy of 0.68, sensitivity of 0.8 and a precision of 0.64.

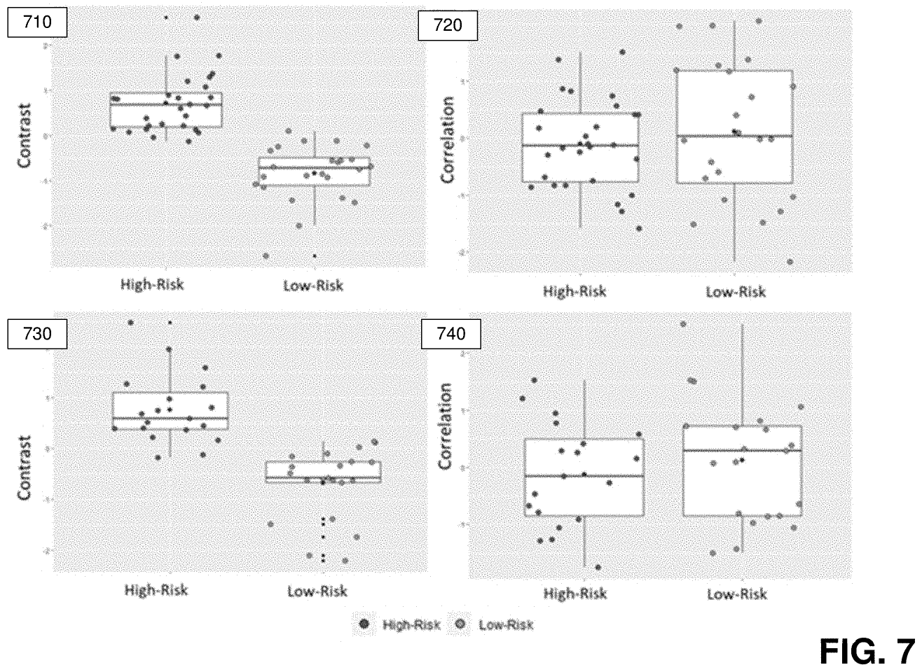

Referring to FIG. 7, illustrated are box plots showing the distribution of each feature in the prognostic signature for high-risk and low-risk patients in the training cohort (710, 720) and the validation cohort (730, 740), in connection with various aspects discussed herein.

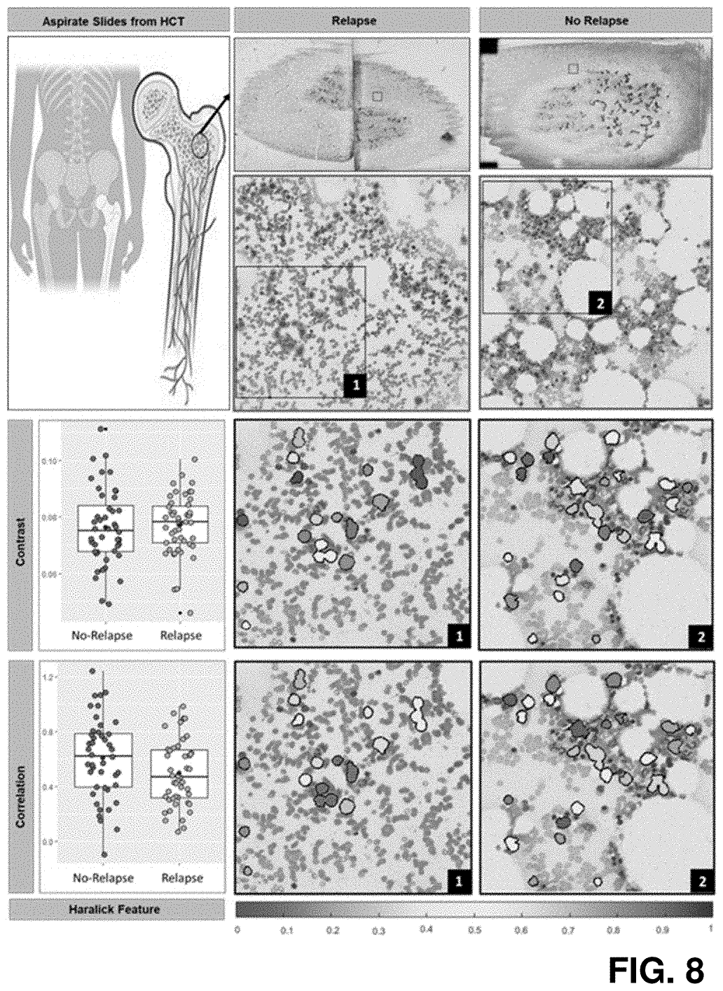

Referring to FIG. 8, illustrated are example images showing the two top LASSO features visualized between relapse and no-relapse patients, in connection with various aspects discussed herein. As the violin plots also reflect, the Haralick contrast feature has higher values on relapse patients compared to no-relapse patients, while for the Haralick correlation feature, no-relapse patients have higher values.

FIG. 8 illustrate the discriminability of the myeloblasts Haralick contrast and correlation features for representative no-relapse and relapse patients. There is higher textural pattern disorder or heterogeneity within myeloblasts of a relapse patient for Haralick contrast feature and lower values within myeloblasts of a relapse patient for Haralick correlation feature as compared with myeloblasts of a no-relapse patient. This trend is also reflected in the box and violin plots of the Haralick contrast and correlation features, illustrated at the bottom of FIG. 8.

Experiment 3: Myeloblast Texture Features are Associated with RFS in AML

The optimum cut-off value for the PRS was found to be the mean PRS in Sₜ, and patients were stratified into high- and low-risk groups based on this value. A univariate Cox regression analysis developed using pathomic features indicated that PRS was significantly negatively associated with RFS in Sₜ (hazard ratio=2.38, 95% confidence interval=1.43-3.95, p=0.0008) and Sᵥ (hazard ratio=1.58, 95% confidence interval=1.01-2.45, p=0.04). The corresponding Kaplan-Meier survival curves showed a significant difference in RFS between patients with low and high PRS (Sₜ: P=0.0008, Sᵥ: P=0.04).

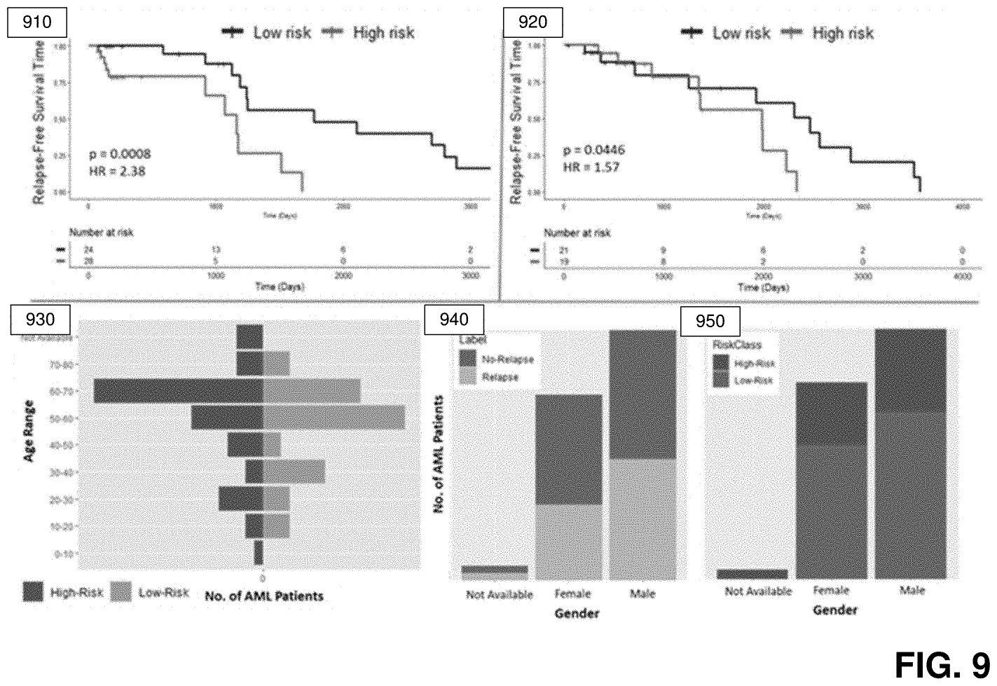

Referring to FIG. 9, illustrated are Kaplan-Meier survival curves for Sₜ and Sᵥ, in connection with various aspects discussed herein. FIG. 9 shows the Kaplan-Meier curves of the high-risk (red) and low-risk groups (blue) in (at 910) the training set (HR=2.38, 95% CI=1.43-3:95, and p=0.0008), and (at 920) validation set (HR=1.58, 95% CI=1.01-2.45, and p=0.04). 930 shows how many high-risk and low-risk patients there are in different age ranges. 940 and 950 show the female and male patients distribution in different groups. These results highlight that the PRS is not only able to prognosticate RFS but also brings complementary information to discriminate HCT outcomes (relapse vs no-relapse) when combined with a LDA classifier.

A multivariable Cox regression model indicated that PRS was the only biomarker associated with RFS in training set (PRS: hazard ratio=2.38; 95% CI, 1.37-4.12; P=0.001; Age: hazard ratio=1.0; 95% CI, 0.98-1.03; P=0.71; Gender: hazard ratio=0.95, 95% CI, 0.36-2.50; P=0.91; C-index=0.74).

### Introduction

Allogenic hematopoietic stem cell transplant (HCT), a last resort curative therapy, is an effective post-remission consolidation treatment for patients with acute myeloid leukemia (AML). However, at least, 40% of patients relapse following HCT. Although myelosuppressive chemotherapy is used to reduce the risk of relapse, it has substantial side effects and is therefore not appropriate for all patients. Therefore, timely prediction of relapse is crucial to direct chemotherapy to high-risk patients only.

Traditionally, manual counting of the myeloblasts on aspirate smear slides by hematopathologists is used to discover which patients are relapsing post-HCT. However, this method is time consuming, susceptible to inter-reviewer variability, error-prone and may also fail to very well distinguish relapse patients while high-risk cytogenetics can better predict relapse. Aside from prognostic factors, such as relevant molecular and cytogenetic aberrations, analysis of cells of routine cytological slide images reveals crucial information on cell physiology. The significance of cytologic interrogation of cells in different types of leukemia is also approved in other studies. Textural and morphological differences in cells offers a rough valuation of complexity in chromatin pattern and thus allows capturing information about how patients respond to treatment. As an example, Auer rods or cytoplasmic granules are reddish, linear structures composed of fused primary granules that may exist in leukemic myeloblasts and their presence indicates myeloid malignancy which may lead to resistance to treatments or ultimately relapse. Laboratory diagnosis of hematological disorders are also generally based on evaluation of characteristics of blood cell chromatic pattern in peripheral blood smears and bone marrow since the chromatin pattern especially in the nucleus is related to cell function. Therefore, interrogating myeloblasts shape and texture features helps to make better decision support tools for prognosticating relapse following transplantation.

Previous work on predicting probability of relapse in AML patients focused on traditional visual (or manual) blast counts and clinical markers (e.g., cytogenetic risk stratification). The first example study went beyond these markers, identifying morphological and textural patterns in myeloblasts which were predictive of post-HCT relapse in AML patients.

In the first experiment, it was found that, in agreement with a prior study, myeloblast count was not a diagnostic or prognostic feature on its own. This may in part be due to imperfections in the myeloblast segmentation model, yet the findings contribute to the body of work suggesting that textural and morphological features are much more prognostic of outcome than simple myeloblast counts.

Additionally, it was found that features of myeleblast morphology and chromatin texture heterogeneity were prognostic of relapse, with higher texture heterogeneity and a more irregular boundary shape being associated with increased risk of relapse. The features that characterize chromatin pattern heterogeneity and complexity have been associated with cytoplasmic and membranous protein expression and might reflect cell maturation and thus disease aggressiveness. That myeloblasts with higher FD values were associated with elevated risk is consistent with previous studies that found an association between higher FD values in malignant cells in AML patients, reflecting a further breakdown of cellular regulation and higher degree of chaos. Myeloblast shape and chromatin pattern may therefore reflect the sum of interactions leading to cell phenotype, providing a potential basis for its prognostic utility.

Results from the third experiment showed that the textural morphological measures are not only prognostic of relapse post-HCT but also associated with the relapse-free survival of the AML patients post-transplant. These features are diagnostic, prognostic, and consistent with previous work in which cell chromatin pattern heterogeneity and complexity reflected DNA methylation patterns related to patient survival. Other studies found that increases in cell nucleus boundary heterogeneity and shape complexity together with emphasized roughness (high FD value) of cell surface in leukemia patients were associated with clinical response to therapy. In the first example study, patients with a smoother chromatin texture and less speckling were more likely to respond to treatment while patients with higher contrast on their myeloblast chromatin pattern mostly experienced AML relapse post-HCT.

The findings are consistent with previous reports that the textural and FD features of fixed and stained cells can be effectively used for discrimination between cells and subsequently between patients with different treatment outcomes. The computational features used in the first example study characterize sub-visual attributes of myeloblasts across aspirate slides that correspond to traits of myeloblast appearance and chromatin texture that are biologically known and interpretable. This property is guaranteed by the feature construction process, where features were designed to quantify characteristics of the cells as described by hematopathologists. This is unlike deep learning approaches where the features are extracted in an unsupervised manner and do not necessarily have a well-informed biological rationale.

Limitations to the first example study include the small size of the validation cohort and the entire dataset was collected from a single institution. Various embodiments can address these limitations.

In the first example study, by analyzing machine extracted features from digitized bone marrow cytological images of AML patients post-HCT, an automated method which can stratify patients by relapse risk using Wright-Giemsa stained aspirate slide images was demonstrated.

### Example Study 2: Computationally Derived Cytological Image Markers for Predicting Risk of Relapse in Acute Myeloid Leukemia Patients Following Bone Marrow Transplantation

The following discussion provides example embodiments in connection with a second example study involving constructing, training, and/or employing models that can automatically segment myeloblasts and/or determine a risk of relapse post-transplant for AML based on features of digitized WSI(s) of bone marrow aspirate.

### Abstract

Allogenic hematopoietic stem cell transplant (HCT) is a curative therapy for acute myeloid leukemia (AML). Relapse after HCT is the most common cause of treatment failure and is associated with poor prognosis. Early identification of which patients are at elevated risk of relapse may justify use of aggressive post-HCT treatment options, potentially preventing relapse and treatment failure. The goal of the second example study was to predict relapse after HCT in AML patients using quantitative features extracted from digitized Wright-Giemsa stained post-transplant aspirate smears. 39 aspirate specimens were collected from a cohort of 39 AML patients after HCT, of which 25 experienced relapse, while 14 did not. The approach comprised the following main steps. First, a deep learning model was developed to segment myeloblasts, a cell type in bone marrow that accumulates and characterizes AML. A total of 161 texture and shape descriptors were then extracted from these segmented myeloblasts. The top eight predictive features were identified using a Wilcoxon rank sum test over 100 iterations of 3-fold cross validation. A model was subsequently built employing these features and yielded an average area under the receiver operating characteristic curve of 0.80±0:05 in cross validation. The top eight features include four Haralick texture features and four fractal dimension features. The texture features appear to characterize chromatin patterns in myeloblasts while the fractal features quantify morphological irregularity and complexity of myeloblasts, in alignment with findings previously reported for AML patients post-treatment.

### 1. Introduction

Acute myeloid leukemia (AML) originates in the blood-forming cells of the bone marrow. AML is characterized by the presence of an excessive number of immature white blood cells, called myeloblasts or more commonly blasts. Myeloblast accumulation in the blood and bone marrow reduces the space for healthy white blood cells (WBCs), red blood cells, and platelets. As a result, patients may experience infection, anemia, or poor blood clotting. Therefore, identifying myeloblasts and assessing their quantity plays a key role in diagnosing AML. Allogenic hematopoietic stem cell transplant (HCT) is a curative therapy for AML, but is associated with significant co-morbidities due to graft-versus-host disease and immunosuppression. 40%-80% of HCT patients relapse following treatment. Myelo-suppressive chemotherapy is used to prevent post-HCT relapse, but can cause various side effects due to the decrease in bone marrow activity. Post-HCT chemotherapy can reduce the risk of relapse, but has substantial side effects and is therefore not appropriate for every patient. Timely relapse prediction after HCT could enable relapse-reducing chemotherapy to be directed, instead, to high-risk patients while allowing low-risk patients to avoid intensive therapy and the resulting side effects.

The current gold standard for early detection of relapse post-transplant is pathologic review of bone marrow aspirate and marrow to assess blast proliferation, as blast quantity is closely linked to AML aggressiveness. This process requires a hematopathologist to count roughly 200 random cells from a bone marrow aspirate specimen and determine if 5% or more of the cells are blasts, which would signal relapse. Intra- and inter-observer variation, the use of only a small proportion of the cells, and the limits of human perception mean that current strategies for assessment of the risk of relapse from the slides are inadequate and sub-optimal. This also suggests that computational interrogation of the cytology images could enable extraction of sub-visual features that may go above and beyond those based off visual identification of the slide images and thus may potentially yield independent prognostic and predictive information regarding AML patients.

Examination of nuclei of routine cytologic slides discloses vital information on cell physiology that is independent of other prognostic factors, such as relevant cytogenetic aberrations. Previous studies also assert the importance of cytologic analysis of cells in different types of leukemia. Textural and morphological variations in cells provides a rough estimation of chromatin rearrangement complexity and therefore enables capturing information about the patients' response to therapy. Haralick proposed texture features to be extracted from Gray Level Cooccurrence Matrices (GLCM). Haralick texture features quantify heterogeneity on the cell surface. Fractal dimension (FD) analysis is a mathematical construct related to self-similarity of an object and describes relevant design principles that underlie living organisms. FD features can properly characterize the complexity and irregularity of microscopic anatomic structures and demonstrate the fractal nature of chromatin in histologic and ultrastructural sections. With the help of computerized image analysis, texture and shape features can be extracted that characterize the complexity of the chromatin architecture in myeloblasts. The second example study sought to evaluate if quantitative descriptors of texture patterns contained within the blasts and the morphological complexities of the blasts could help in predicting relapse and prognosis in AML treated patients.

The remainder of the second example study is organized as follows. Section 2 discusses building an automated blasts segmentation model. Texture and shape features were then extracted from blasts segmented on random, non-overlapping tiles. Section 2.4 describes the experiments for identifying most discriminating features and how classification was performed. Sections 3 demonstrates experimental results and Section 4 presents discussion. Finally, concluding remarks and future directions are described in Section 5.

### 2. Methods and Experimental Design

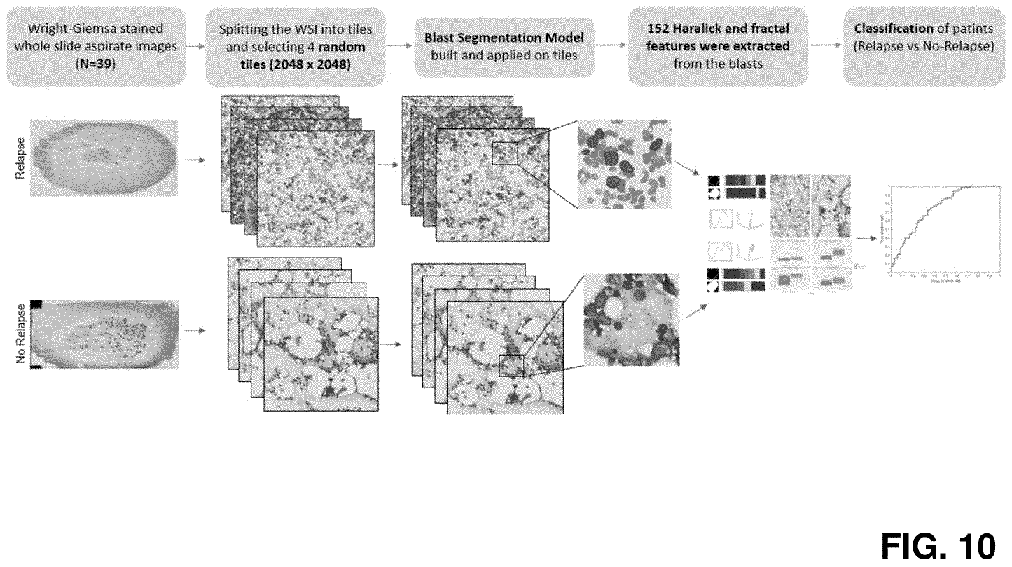

Referring to FIG. 10, illustrated is a flowchart showing the image analysis steps for identifying blasts and then using the shape and appearance and arrangement of the blasts on aspirate cytological images to predict response to HCT, in connection with various aspects discussed herein. FIG. 10 presents the overall methodology of the second example study, including tiling, blasts segmentation, feature extraction, and classification.

### 2.1 Patient Dataset

Bone marrow Wright-Giemsa aspirate slides were collected from n=39 AML patients six to eight weeks after HCT. Of these patients, 25 experienced relapse. All slides were digitized at 40× magnification. Four random non-overlapping 2048×2048 pixel tiles were selected from each digitized aspirate image (overall 156 2048×2048 pixel tiles).

### 2.2 Automated Blast Segmentation

The blast segmentation framework consisted of two training steps based on conditional generative adversarial networks (cGAN). Two sets of 256×256 pixel patches were constructed for WBC segmentation and myeloblast segmentation. There were 122 annotated 256×256 pixel patches for WBC segmentation, of which 22 random patches were used only for testing. Patches were annotated for WBCs by a trained technician. To train the myeloblast segmentation model, 184 annotated 256×256 pixel patches were used, of which 14 random patches were used as an independent test set. Patches were annotated for myeloblasts by a hematopathologist.

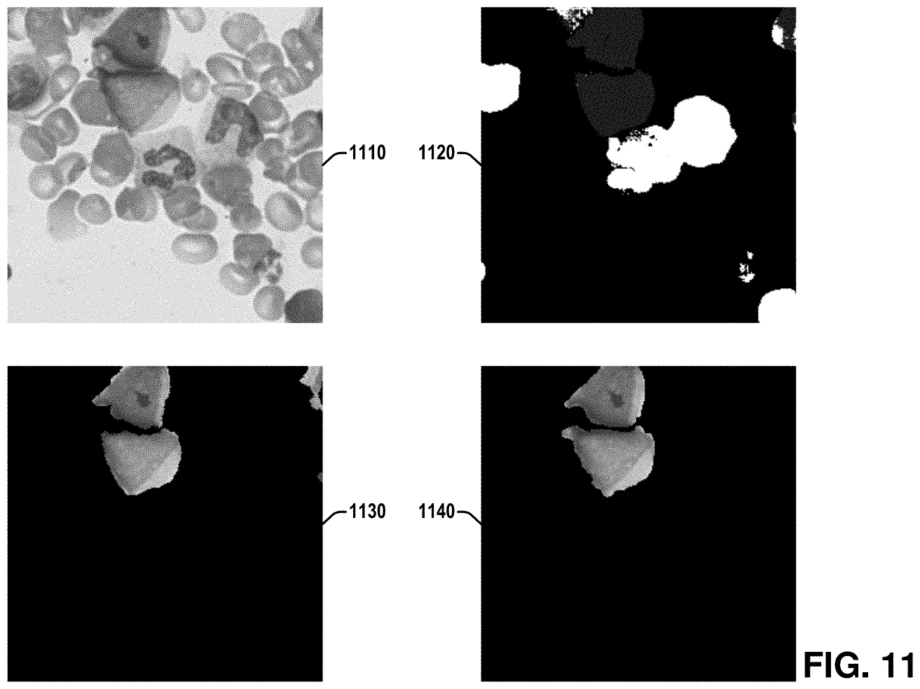

The first training step aimed to segment all WBCs against background, red blood cells, and artifacts. This step leveraged the similar appearance of blasts and other WBCs to produce a segmentation of candidate objects. In the second step, the WBC segmentation model was fine tuned for blast detection. This segmentation framework achieved a mean dice coefficient of 0.953 in WBC segmentation and 0.912 in blast segmentation. Referring to FIG. 11, illustrated are example images showing the blast segmentation results, in connection with various aspects discussed herein. FIG. 11 shows Blast segmentation results on an image from the test set. Shown is the original image (1110), the output of segmentation results (1120) in WBC identified regions (in white) and blast identified regions (in blue). 1130 shows further pruning to eliminate the WBC components and isolate the blasts alone and 1140 shows the corresponding ground truth annotation for the blast for the same region of interest shown in 1130 but from a hematopathologist.

### 2.3 Feature Extraction

Two categories of features were then extracted from each segmented blast. The first category comprised thirteen Haralick texture features and the second set included 21 one dimensional (1 D) and two dimensional (2D) fractal features to assess morphological and textural changes of blasts within aspirate slides. To measure 1D fractal dimension, each blast was converted to a time series using distance of each pixel on its boundary from the blast's center. Then the obtained time series was embedded to state space reconstruction based on mutual information method. The 2D fractal dimension is measured using box-counting method from the blasts' images and masks. The mean, median, standard deviation, and skewness of each feature was calculated across all myeloblasts on the tile. For every patient, the mean of these values across all four tiles was computed to arrive at a total of 161 features for each patient.

### 2.4 Experiments

In order to build a well-structured relapse prediction model, experiments were performed for identifying top discriminating features and evaluating their predictive performance using different classification methods.

### 2.4.1 Experiment 1: Evaluating Feature Discriminability

To identify the top discriminating features to be employed in training a prediction model, feature selection was performed in a cross-validation setting. From each feature group (52 Haralick texture features and 109 FD features), a set of top features was separately identified across 100 iterations of 3-fold cross validation. In each training fold, four features were selected by the Wilcoxon rank-sum test. Following cross-validation, the four features most frequently selected features across cross-validation folds were identified as the overall top feature set for further evaluation and visualization. The features can be compared by their different expressions in box plots.

### 2.4.2 Experiment 2: Predicting Relapse

Three models were trained for predicting AML relapse. The first model used Haralick features extracted from blast texture to train a random forest model. The second model used fractal features extracted from blast surface texture and boundary shapes to train a linear discriminant analysis (LDA) model. These models were trained and evaluated across 100 iterations of 3-fold cross validation. Each model iteration was trained utilizing the four features chosen by the Wilcoxon rank-sum test only within the respective training fold.

A third model, a LDA model incorporating the most frequently selected features from both feature families (i.e., four Haralick features and four fractal features), was again trained across 100 iterations of 3-fold cross validation. Model performance in the prediction of AML relapse was evaluated by the area under the receiver operating characteristic curve (AUC).

### 3. Results

### 3.1 Experiment 1: Evaluating Feature Discriminability

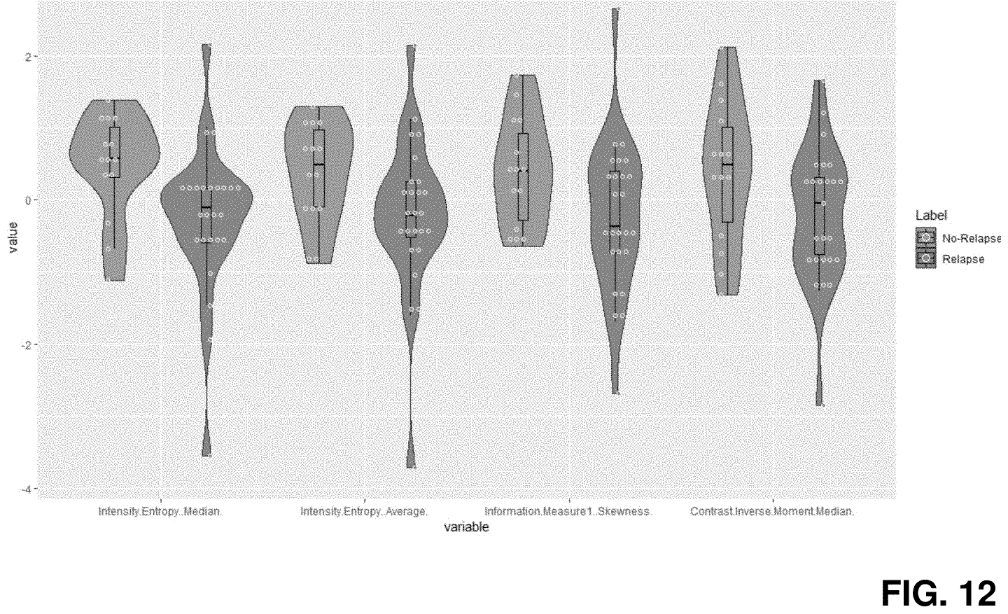

The most frequently selected features are listed in Table 3, below. Referring to FIG. 12, illustrated are violin plots of the four top discriminating features in predicting relapse across the entire dataset of n=39 images, in connection with various aspects discussed herein. The relapse group shows lower intensity entropy, information measure 1 and contrast inverse moment.

TABLE 3
Eight top discriminating features selected most frequently in
100 iterations of 3-fold cross validation

| No. | Haralick Features | Fractal Dimension Features |
|---|---|---|
| 1 | Intensity Entropy | Entropy in time series |
| | (Median) | |
| 2 | Intensity Entropy | Skewness of 2D fractal dimension |
| | (Average) | of blast texture |
| 3 | Information | The least value in 2D Fractal |
| | Measure1 | dimension of |
| | (Skewness) | blasts texture (Average) |
| 4 | Contrast Inverse | The least value in 2D Fractal |
| | Moment | dimension of |
| | (Median) | blasts texture (Maximum) |

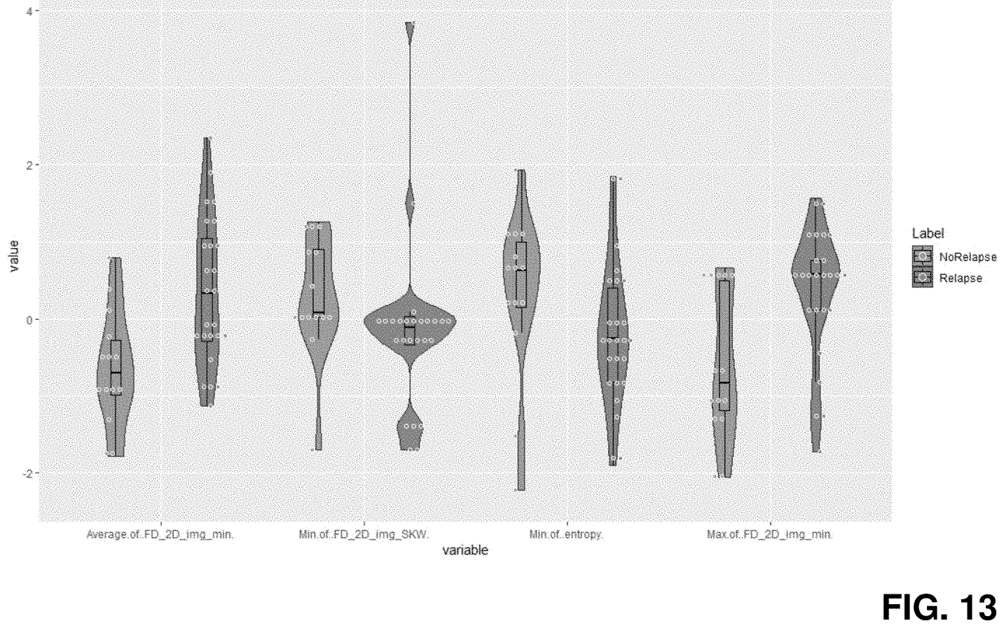

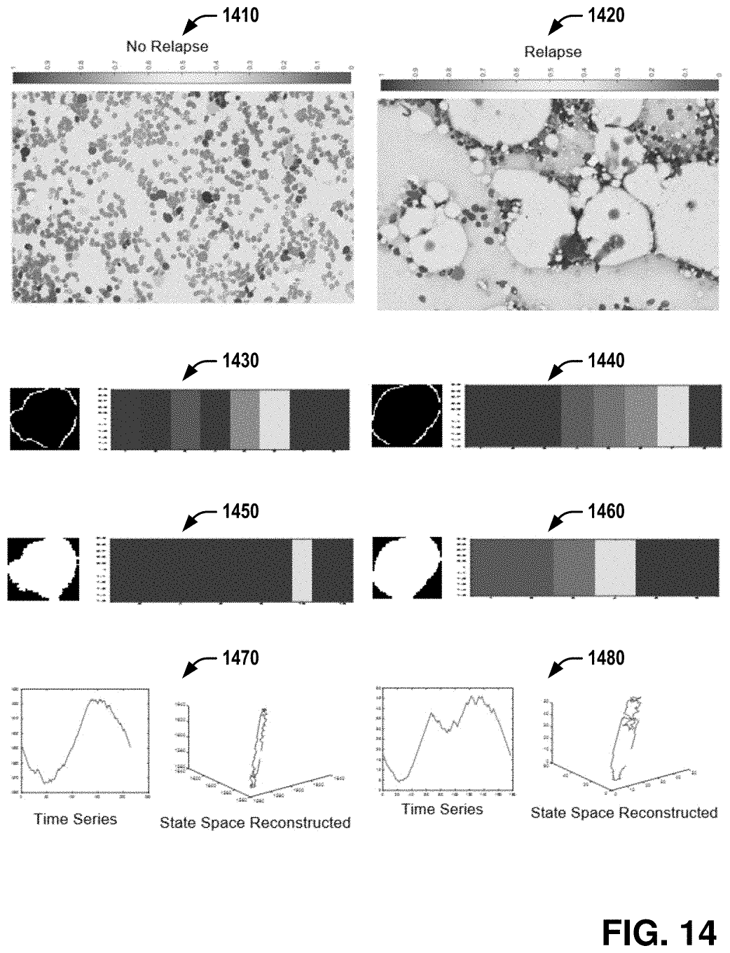

Referring to FIG. 13, illustrated are violin plots of the four top fractal features in predicting relapse across the entire dataset of n=39 slides, in connection with various aspects discussed herein. FIGS. 13 and 14 depict the distributions of the most frequently selected features in violin plots for Haralick and fractal features, respectively. Consistent across these feature families, patients who do not relapse (the no-relapse group) consistently demonstrate elevated expression of features characterizing complexities in blasts' shape and texture.

Referring to FIG. 14, illustrated are images and graphs showing differences in fractal feature expression between patients who will relapse from those who will not, in connection with various aspects discussed herein. FIG. 14 shows visualization of 2D fractal dimension characterizing chromatin pattern and morphological structural complexity of the blasts in a (1410) no-relapse patient versus a (1420) relapse patient and how warmer colors are more prevalent in no-relapse showing higher values of the entropy in time series. The heat map of 2D fractal dimension with different scales of boxes in box-counting method corresponding to boundary of a blast is shown in a (1430) no-relapse and a (1440) relapse patient. The heatmap of 2D fractal dimension over chromatin pattern of the segmented blast is shown in a (1450) no-relapse patient and a (1460) relapse patient. Time series of blast boundary based on distance of each pixel from blast's centroid is shown in a (1470, left) no-relapse and a (1480, left) relapse patient respectively. State space reconstructed from obtained time series using mutual information method is shown in a (1470, right) no-relapse and a (1480, right) relapse patient respectively. The heat maps in FIG. 14 illustrate that chromatin pattern of blasts on a no-relapse patient slide has more heterogeneity comparing to that of a relapse patient, confirming that increased textural and morphological complexity of blasts is associated with more favorable patient outcomes. The heat maps and box plots of FD features that characterize heterogeneity have higher values in the no-relapse group.

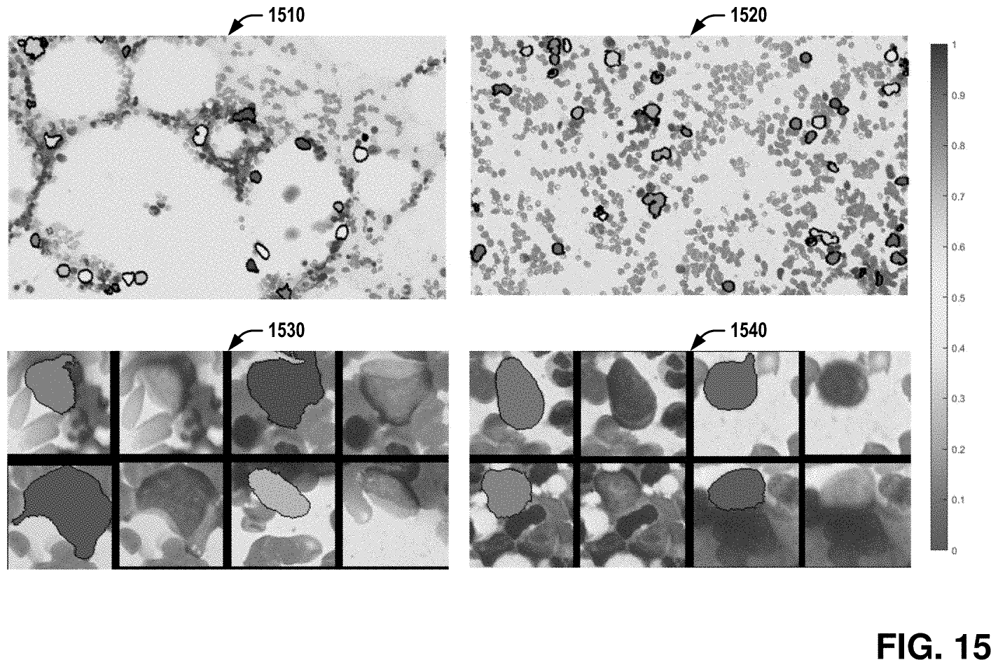

Referring to FIG. 15, illustrated are example images that visualize the differences of intensity entropy values on blasts in relapse and no-relapse patients, in connection with various aspects discussed herein. Lower entropy (both in Haralick and FD features), indicate patients poor response to therapy (relapse group). FIG. 15 shows visualization of intensity entropy on blasts of a (1510) no-relapse and a (1520) relapse patient, respectively. 1510 and 1520 demonstrate two representative tiles where the blasts are segmented. 1530 shows representative blasts from the no-relapse group, where warmer colors confirm higher values of intensity entropy. 1540 shows blasts from relapse patients, which were found to have lower intensity entropy values, shown as colder colors.

### 3.2 Experiment 2: Predicting Relapse

Across 100 iterations of 3-fold cross validation, the mean AUC of the three classifiers developed in Section 2.4 in predicting the likelihood of relapse was 0.63±0.10, 0.67±0.08, and 0.80±0.05, respectively, where the first model was only based on Haralick texture features, the second model applied fractal texture and shape features and the third model benefited from the best features of the first and second models.

### 4. Discussion

Previous work on predicting likelihood of relapse in AML patients was focused on manual blast counts and clinical markers (e.g., cytogenetic risk stratification). The second example study developed an automated myeloblast segmentation algorithm and extracted features of blast chromatin texture and shape from post-HCT aspirate images. These features were then used to create a model which would predict which patients would experience relapse of AML.

FD analysis is a scale-invariant measurement method that enables mathematical explanation of the ruggedness of natural surfaces. In the field of medicine, fractal analysis has been applied to pathology, anatomy, and medical imaging to characterizes shapes, textures, contours, the heterogeneity and complexity of objects in an image. The employed Haralick and FD features of chromatin texture were designed to capture changes in blast chromatin structure and fractal morphometry, which has been associated with cytoplasmic and membranous protein expression and may reflect cell maturation and thus disease aggressiveness. The findings of the second example study that these features are predictive is consistent with previous work in which cell chromatin pattern heterogeneity and complexity reflected DNA methylation patterns related to patient survival. Other studies support these results, explaining that the increase in cell chromatin pattern heterogeneity and complexity together with emphasized roughness (high FD value) of cell surface were associated with good clinical response to therapy. This finding strengthens the assumption that a higher FD reflects a healthier, well-equilibrated biological system. In the second example study, patients with a smoother chromatin texture and less speckling were more likely to have AML relapse post-HCT.

Limitations to these promising initial findings include the small cohort size and lack of an independent validation set. An additional limitation was that the analysis was conducted on select regions of interest on the images as opposed to the entire slide image. Various embodiments can address these limitations.

### 5. Conclusion

The second example study demonstrated that features of chromatin texture and blast shape mined from myeloblasts in digital aspirate slide images may predict likelihood of relapse in AML patients after HCT. Quantitative descriptors of blast texture and shape, potentially influenced by chromatin appearance and morphological structural complexity, were found to differ between patients who relapsed post-transplant and those who did not. These findings support the use of various embodiments for automated analysis methods for identifying patients who may benefit from additional therapy following transplant.

### Example Study 3: Automatic Myeloblast Segmentation in Acute Myeloid Leukemia Images Based on Adversarial Feature Learning

The following discussion provides example embodiments in connection with a third example study involving constructing, training, and/or employing models that can automatically segment myeloblasts and/or determine a risk of relapse post-transplant for AML based on features of digitized WSI(s) of bone marrow aspirate.

### Abstract

Diagnosis of hematological malignancies such as Acute Myeloid Leukemia (AML) demands reliable detection and counting of immature white blood cells called myeloblasts on bone marrow aspirate and biopsy slides. Experienced hematopathologists typically spend many hours manually reviewing blood cells and performing microscopic morphological examination on the myeloblasts to help determine the best medication and therapy for patients. While manual counting is considered the gold standard, the process is time-consuming, monotonous, and difficult to standardize due to high intra- and inter-observer variation. Machine learning algorithms coupled with deep learning techniques provide a highly promising technology for developing a reliable automated counter. The end-to-end segmentation model based on Convolutional Neural Network (CNN) can achieve better segmentation performance in the segmentation of pathological images. However, myeloblasts have very similar texture attributes to other types of white blood cells and this challenges the general segmentation models. The third example study developed a conditional Generative Adversarial Network (cGAN)-based segmentation model (AMLcGAN) to efficiently segment myoblasts in AML images. Validation on 204 pathological images with the size of 256×256 shows that the mean pixel accuracy of AMLcGAN is as high as 96.2%, and the mean dice coefficient is 87.1%. At the same time, AMLcGAN was compared with three other segmentation models, and the experimental evaluation results show that the performance of AMLcGAN is promising. In various embodiments, the approach can be used as an aid for the examination of cells on aspirate smears of AML patients that is usually performed by a human expert.

### 1. Introduction

Leukemia is a group of blood cancers starting in bone marrow and affecting the white blood cells (WBC) or leukocytes. In leukemia, abnormal and immature WBCs that are unable to fight infection overproduce and outnumber the healthy WBCs. It is critical to detect the disease early enough to manage proper treatment. Abnormal WBCs or myeloblasts play a significant role for hematologists in the diagnostic process. Hematological diagnostics such as early and fast detection of the leukemia type, greatly helps in providing the appropriate treatment and depends on pathological review of aspirate and biopsy slides and microscopic examination and classification of blood cells known as complete blood count (CBC). While manual counts are considered the gold standard, they are labor intensive, time consuming, and subject to bias. Examination of the bone marrow is a critical process in the hematology field for many diseases related to blood and bone marrow and it is a common procedure in laboratories. As part of this examination, a myeloblast count is obtained by microscopy on Wright-Giemsa stained bone marrow aspirate smears. In myeloid malignancies such as AML and myelodysplastic syndromes, the disease defining criteria are based on cutoff percentages of myeloblasts. A trustworthy automated counter has yet to be developed, largely due to the intrinsic intricacy of bone marrow specimens. Deep CNNs have proven very successful in the field of natural image analysis. Recently, CNNs have been successfully applied to various medical imaging tasks, including cell segmentation, nuclei segmentation, and tissue type segmentation. This motivated the third example study's application of CNNs to the segmentation of myeloblasts on cytopathology images to build an automatic blast counting tool. Compared with the manual segmentation models, the CNN-based segmentation model shows more promising segmentation performance. CNN-based segmentation methods are mainly divided into two types according to the segmentation process: a segmentation method based on patch-wise classification and an end-to-end segmentation method based on semantic segmentation. The method based on patch-wise classification can get higher local classification accuracy, but it is time-consuming to segment big image blocks; furthermore, and this method is based on the features of local patches, so it cannot outline the boundaries of the objects. The end-to-end model based on semantic segmentation can obtain the semantic information of bigger patches to better outline the boundaries of the objects, and the segmentation speed is faster. However, training the end-to-end segmentation model is more difficult, and the classification accuracy of the target with complex texture structure is low. In order to overcome these challenges, the third example study employed a cGAN-based segmentation architecture named AMLcGAN to achieve the automated semantic segmentation of myeloblasts and other types of WBCs in blood smears.

### 1.1 Related Work

The generative adversarial nets (GAN) theory was proposed by Goodfellow et al in 2014 to generate fake images. Later it was successfully used in many image processing fields. Later, the model Pix2pix was proposed, based on the Conditional Adversarial Generation Network (cGAN), which effectively solves the problem of image translation, and finds a new method of image segmentation with cGAN. Compared with the end-to-end semantic segmentation model, the biggest difference is that Pix2pix has a trainable loss function. This is very important in the actual image segmentation task, because a predefined loss function cannot accurately measure the distance between the model output and the label mask in all image segmentation tasks. A trainable loss function allows the segmentation model to be better trained. In cGAN, the optimization objective adversarial function of the model is given by formula (1):

$${min}_{G} {max}_{D} L ( D , G ) = {𝔼}_{x ∼ Pdata ( x )} [ log D ( x \vert y ) ] + {𝔼}_{z ∼ Pz ( z )} [ log ( 1 - D ( G ( z \vert y ) ) ) ] \qquad ( 1 )$$

Where G and D represent the generator and discriminator, respectively; x represents the real training data; y represents the label of the true and false data; and z represents the input random noise vector. The loss function of the Pix2pix model consists of two parts: conditional adversarial loss and image pixel loss. The conditional adversarial loss of Pix2pix is given by formula (2):

$${min}_{G} {max}_{D} L ( D , G ) = {𝔼}_{x , y} [ log D ( x , y ) ] + {𝔼}_{x , z} [ log ( 1 - D ( x , G ( x , z ) ) ) ] \qquad ( 2 )$$

In Pix2pix, the generator uses an end-to-end Unet in which the conditional input of the discriminator is the original input image of the generator, and the input random noise z of the generator is meant to increase the diversity of the generated samples. The experiment of the Pix2pix model proves that the model using the adversarial loss has better performance. Compared with ordinary CNN, GAN training is generally more difficult. Inspired by previous work, a feature matching loss was added to the loss function of AMLcGAN, which is defined as in formula (3):

Where d(▪) defines the feature maps of a middle layer of the discriminator. According to the experiments, adding feature matching loss can not only stabilize AMLcGAN training, but also increase its ability to detect myeloblasts.

### 1.2 Contribution

The third example study developed a novel segmentation architecture based on cGAN to segment myeloblasts in blood smear images. Compared with other popular segmentation models based on deep learning, the model developed for the third example study is more promising.

### 2. AMLcGAN

### 2.1 Overall Training Process of AMLcGAN

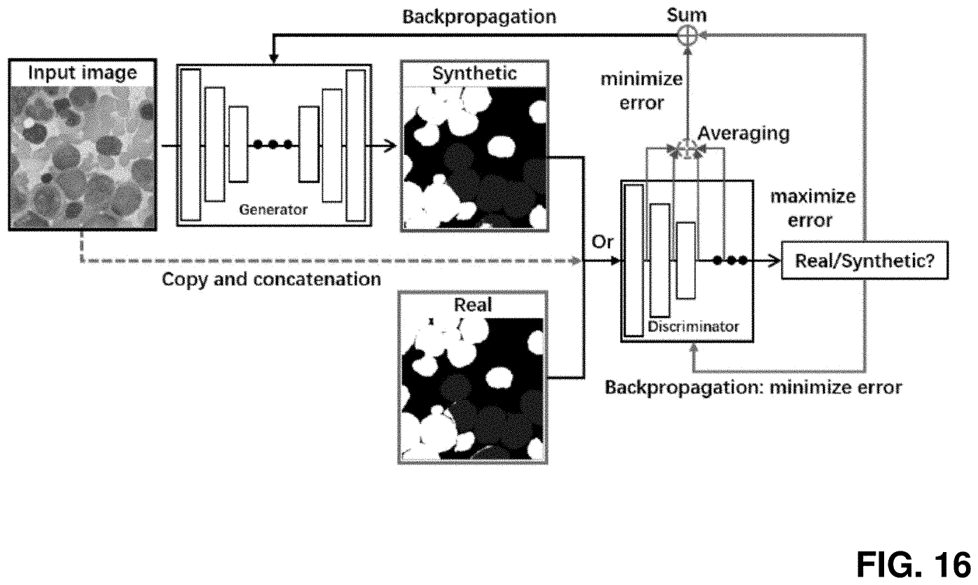

The proposed AMLcGAN comprises a generator and a discriminator within an integrated end-to-end framework, where the generator acts as a segmentation network. Referring to FIG. 16, illustrated is a diagram showing the AMLcGAN training process, in connection with various aspects discussed herein.

In the forward propagation of the generator, the generator is fed a blood smear image, and the generator will output a segmented image. There are two steps in the forward propagation of the discriminator: (1) the original pathological image is cascaded with the target image synthesized by the generator and the target image of the real label to form real and synthetic sample pairs respectively; (2) The real and synthetic sample pairs are input to the discriminator network one-by-one, and the discriminator will output the feature maps of the middle layer and the discriminant probability of the output layer respectively.

In the back propagation of the discriminator, the discriminator will update its own parameters based on the output in order to minimize the discriminant loss of the real or synthetic sample pairs. In the back propagation of the generator, the parameter update of the generator comes from the adversarial loss and the feature matching loss. The adversarial loss is the maximum discriminant loss of the discriminator on the synthetic sample pairs, and the feature matching loss is the error between the deep features extracted by the discriminator on the synthetic or real sample pairs, respectively.

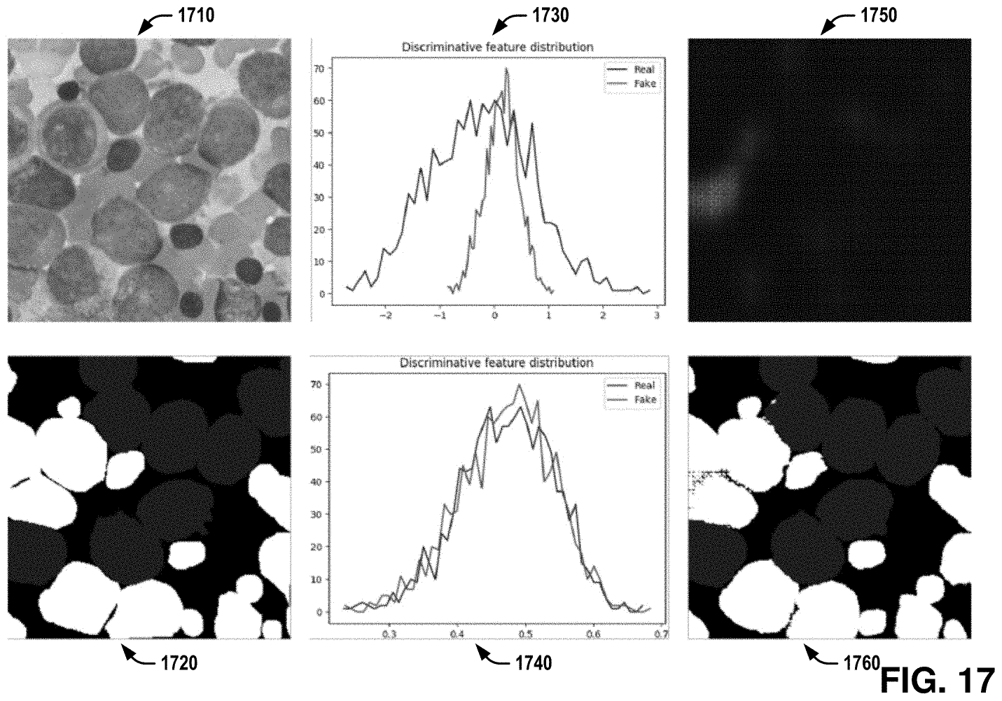

During the training process, the discriminator will continuously update the parameters according to its own discriminant loss to extract more accurate high-dimensional feature expressions of real and synthetic images, while the generator will continuously update the network parameters according to the discriminator's adversarial loss and features matching loss to reduce the error between the outputs and the real samples. The discriminator guides the training of the generator by calculating the error in the high-dimensional feature distribution between the synthesized samples and the real samples, so the discriminator is equivalent to a trainable loss function. Referring to FIG. 17, illustrated are example images and graphs showing the influence of the discriminative feature distribution of the discriminator output on the output of the generator during the training phase, in connection with various aspects discussed herein. FIG. 17 illustrates the relationship between discriminative feature distribution and generator output, showing: at 1710, a 256×256 pathological image; at 1720, the corresponding segmentation label image; at 1730 and 1750, the feature distribution of the discriminator output and the segmentation result of the generator at the beginning of training, respectively; and at 1740 and 1760, the feature distribution of the discriminator output and the segmentation result of the generator at the end of training, respectively.

The discriminator of AMLcGAN discriminates the synthesized samples and the real samples at the patch level. Specifically, the output of the discriminator is a fixed-size patch, and the average value of this patch can be used to obtain the final discriminant probability output. It can be seen from 1730 and 1740 that the discriminator supervises the training of the generator during the entire training process and makes the final output image of the generator and the real target image with the smallest error in the feature distribution.

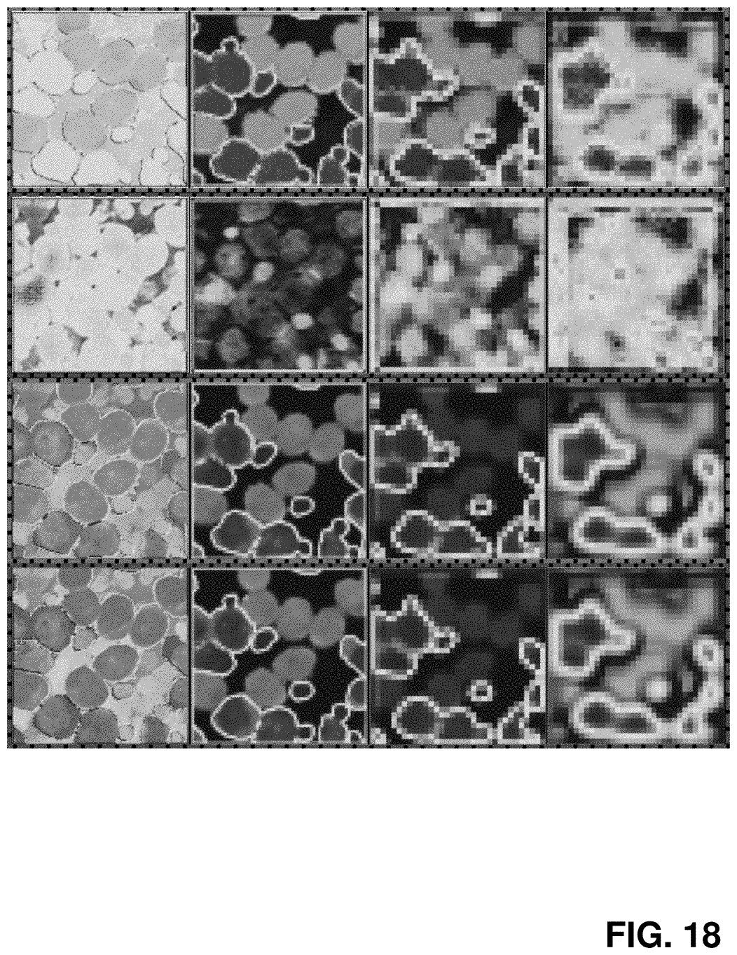

Referring to FIG. 18, illustrated are example images showing discriminator feature visualization, in connection with various aspects discussed herein. From top to bottom, the first two rows are the feature visualization results at the beginning of AMLcGAN training, and the last two rows are the feature visualization results at the end of the training. From left to right, the columns are the output features of each layer of the discriminator from large to small scale. The green dotted frames and the red dotted frames represent the visual features of the input samples of the discriminator that are real image pairs and synthesized image pairs, respectively. During the training process, the visualization results of each layer of the discriminator are as shown in FIG. 18. These visualization results are the mean value of the output feature map of each layer of the discriminator. FIG. 18 uses the same test sample as FIG. 17, and the input of the discriminator is 1710-1720, 1710-1750, 1710-1760 three combined image pairs.

As the training progresses, the discriminator is used to perform feature extraction and feature matching on the synthesized samples of the generator and the truly labeled samples. The output of the final generator will have a minimum loss between the features of the discriminator at multiple scales and the real labeled samples.

### 2.2. Structure of Generator and Discriminator

The generator of AMLcGAN consists of three components: a front-end encoder with three convolutional down-sampling layers, a series of residual blocks, and a back-end decoder with three transposed convolutional up-sampling layers.

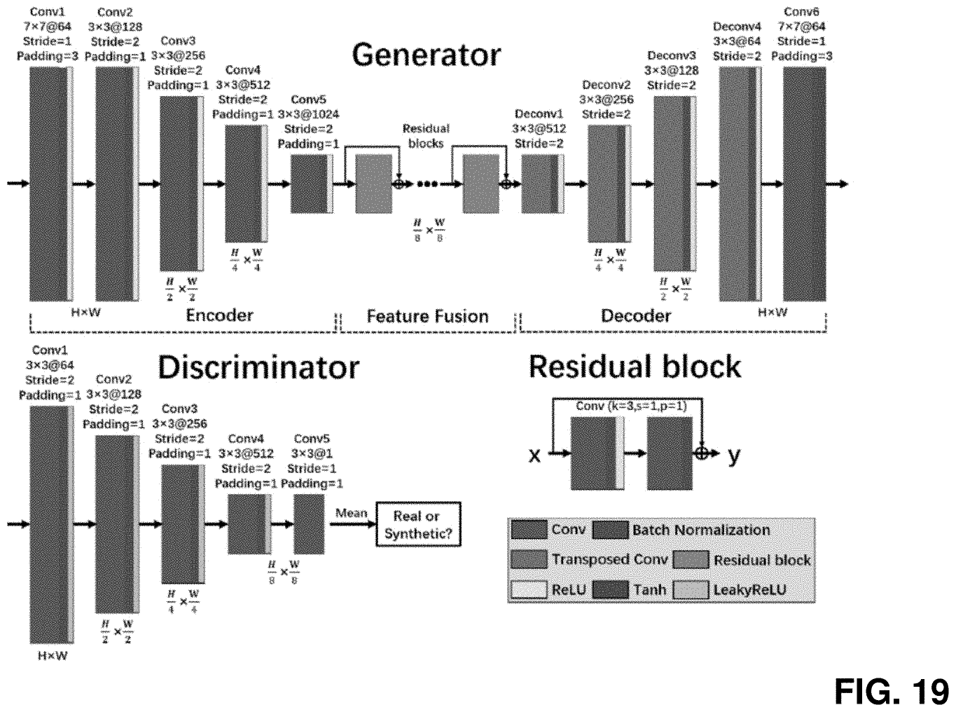

This basic structure has been successfully used in high-resolution neural style transfer image tasks. The convolutional down-sampling encoder and deconvolutional up-sampling decoder avoid the use of pooling layers, so they can better obtain in-depth context-aware expressions and smoothly extract features. A series of residual blocks connect the encoder and decoder for deep feature fusion and translation that is capable of obtaining deep task-aware representation and preserving fine-grained information as well. Referring to FIG. 19, illustrated is a diagram showing the detailed module structures of AMLcGAN, in connection with various aspects discussed herein. FIG. 19 shows the generator and discriminator structure of AMLcGAN (In the residual blocks, the input and output have the same size and the same number of channels). In the generator, except for the input layer and output layer that use 7×7 large convolution kernels, all other convolutional layers and deconvolutional layers use 3×3 small convolution kernels, and each convolution layer is cascaded with a batch normalization layer to ensure the stability of training. At the same time, except for the batch normalization layer in the output layer and the second batch normalization layer in the residual block, all other batch normalization layers use a ReLU activation function to increase the nonlinearity of the model. The generator body comprises 12 residual blocks. In the output layer, a Tanh activation function was used to ensure that the output has pixels in the range [0, 255].

The discriminator and the encoder module in the generator have a similar structure and also contain three convolutional down-sampling layers with the same convolution parameters, so as to ensure that the discriminant features of the discriminator and the encoding features of the generator have the same size, which increases the training stability of the generator. The differences from the generator are that the activation functions in the discriminator all use leakyReLU, the final feature discrimination layer is a single convolutional layer, and the final output is the arithmetic mean of the convolutional layer.

### 2.3. Loss Function

The optimization objective of AMLcGAN consists of two parts: conditional adversarial loss and feature matching loss. Similar to formula (2), the conditional adversarial loss function is given in formula (4):

$${L}_{cGAN} ( G , D ) = {min}_{G} {max}_{D} L ( D , G ) = {𝔼}_{x , y} [ log D ( x , y ) ] + {𝔼}_{x} [ log ( 1 - D ( x , G ( x ) ) ) ] \qquad ( 4 )$$

The difference from formula (2) is that there is no need to increase the diversity of generator, so random noise is not needed as an input. The feature matching loss function is given in formula (5):

$${L}_{fm} ( G , D ) = {𝔼}_{x , y} {\sum }_{i = 1}^{4} [ \frac{1}{4} {‖ {d}^{i} ( x , y ) - {d}^{i} ( x , G ( x ) ) ‖}_{1} ] \qquad ( 5 )$$

Similar to formula (3), dⁱ(▪) defines the feature output of the i-th layer of the discriminator. In the discriminator, in addition to the final discriminative feature output layer, the feature matching loss is calculated for the output features of each convolutional down-sampling layer. In feature matching, the discriminator only serves as a feature extractor without affecting the calculation of formula (5). The final objective loss function is given by formula (6):

$${min}_{G} ( {max}_{D} {L}_{cGAN} ( G , D ) + λ {L}_{fm} ( G , D ) ) \qquad ( 6 )$$

Where λ=10 controls the weight of the contributions of the two terms.

### 3. Experiment

AMLcGAN does not use any pre/post-processing and data augmentation for pathological images. The optimizer used in the generator and discriminator is the Adam algorithm, where the initial learning rate is 0.0002, β1 is 0.5, and β2 is 0.999. The AMLcGAN is implemented on Python 3.7 and Pytorch 1.2.0 library. The mini-batch size during training is 16 and training epochs is 150 using one Nvidia GPU 1080ti with cudnn v7.6 and Intel CPU Core™ i7-4790 @3.60 GHz.

### 3.1. Datasets

The proposed AMLcGAN was trained and evaluated on a data set containing 681 tiles of 256×256 size. These tiles were obtained from Wright-Giemsa aspirate whole slide images of bone marrow of AML patients after bone marrow transplantation at University Hospitals Cleveland Medical Center (UHCMC). All images have Myeloblast (blue) and other types of white blood cells (white) marked by the pathologist at 40× optical resolution. The dataset was randomly divided into a training set (477 images) and a test set (204 images) according to a 7:3 ratio. Then 77 images were randomly selected from the training set as an independent validation set. Note that there is no intersection between the validation set and the training set. As mentioned earlier, none of the images went through any prepossessing operations such as data augmentation and standardization. After each iteration epoch in the training phase of AMLcGAN, the test was forwarded on the validation set and the result produced by the generator was evaluated.

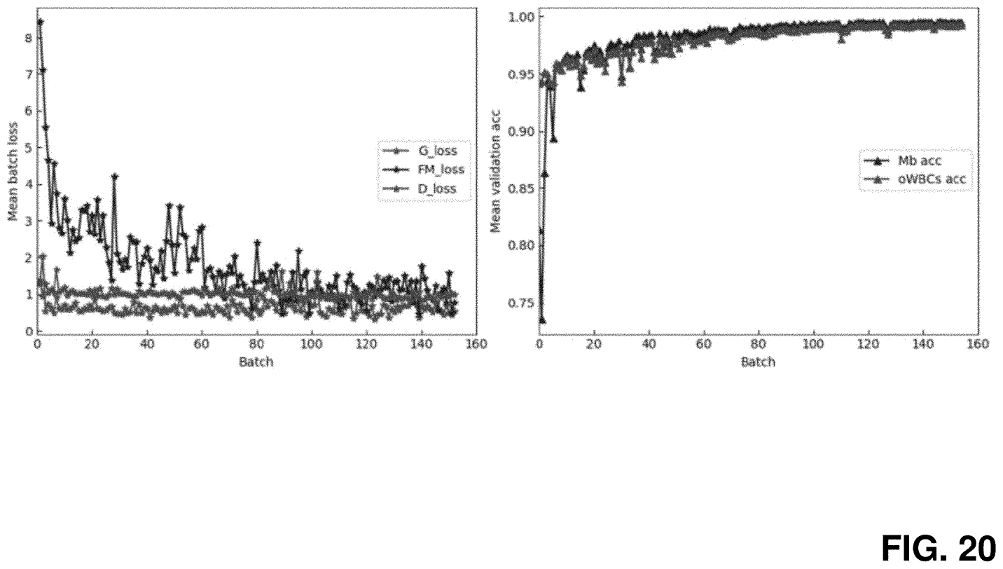

Referring to FIG. 20, illustrated are two graphs showing the average adversarial loss and average feature matching loss of the generator and discriminator in each epoch during the AMLcGAN training process, and the segmentation accuracy of the generator on the validation set after each training epoch, in connection with various aspects discussed herein. Gₗₒₛₛ and Dₗₒₛₛ refer to the average adversarial loss of the generator and discriminator respectively, and FMₗₒₛₛ refers to the feature matching loss.

### 3.2. Result and Analysis

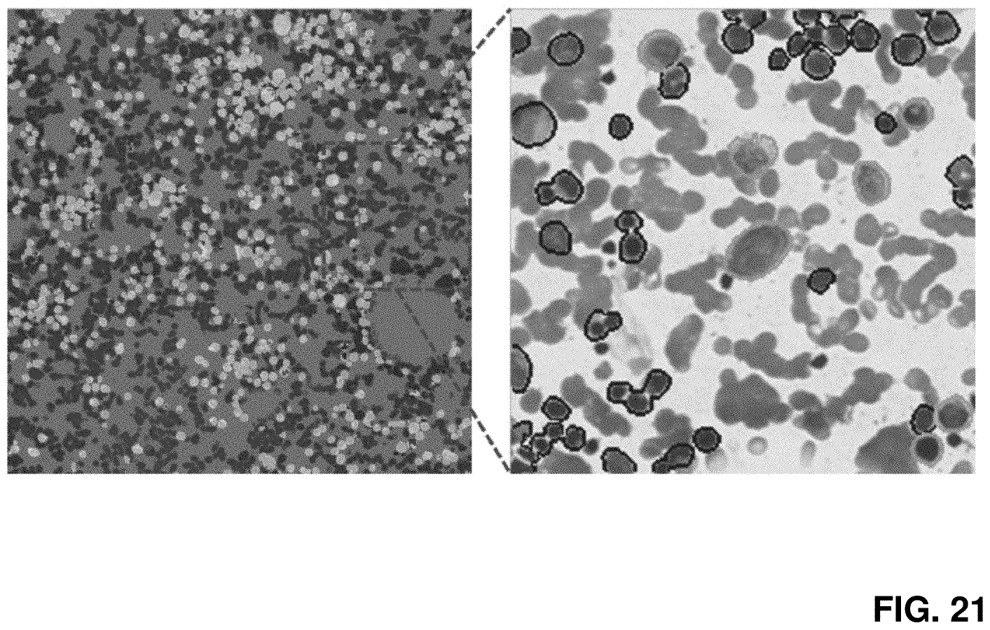

Once the training of AMLcGAN was completed, the pathological images were fed into the generator. The segmentation results of myeloblasts and other types of WBCs could then be directly obtained. Referring to FIG. 21, illustrated is an example image (left) and magnified region (right) showing visualization of segmentation results, in connection with various aspects discussed herein. In FIG. 21, myeloblasts were marked with green curves, and other types of white blood cells were marked with blue curves. Since the generator is an end-to-end structure, the input and output images of the generator have the same size. Considering that both the down-sampling and up-sampling of the feature maps in the generator have a 2× relationship, the input image size of the generator should be equal to 2ⁿ, where n should be a positive integer greater than seven (e.g., 2⁸=256).

At the same time, the segmentation results of Unet, Segnet, and Pix2pix were compared on the independent test set.

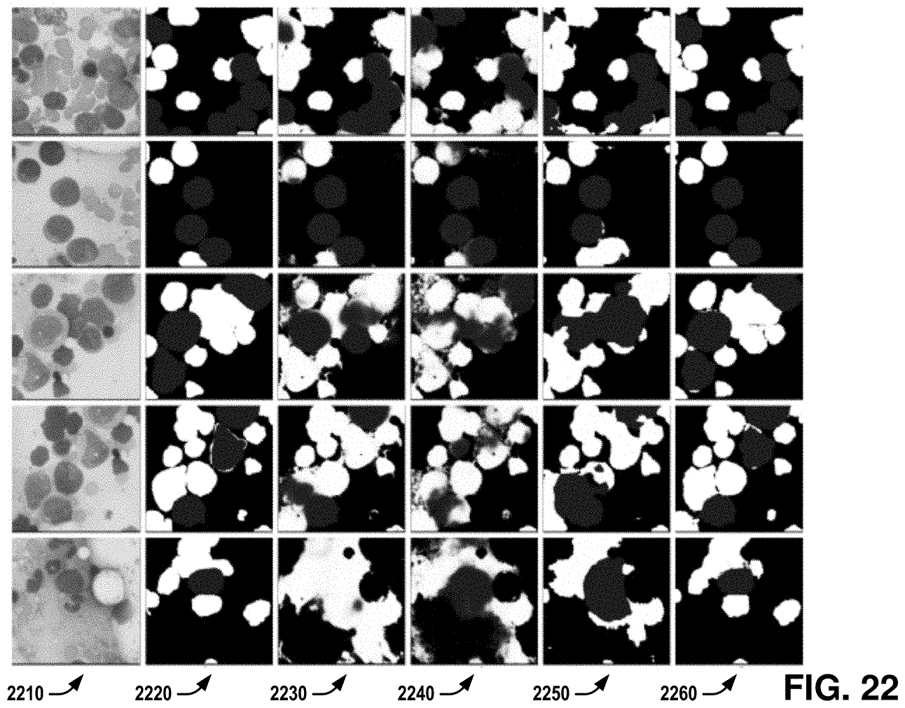

Referring to FIG. 22, illustrated are example images showing comparison results of different segmentation models, in connection with various aspects discussed herein. In FIG. 22, column 2210 shows input images, column 2220 shows label images, column 2230 shows UNet results, column 2240 shows SegNet results, column 2250 shows Pix2pix results, and column 2260 shows AMLcGAN results. FIG. 22 shows that AMLcGAN (2260) can achieve the best segmentation performance, and the segmentation results of myeloblasts and other types of WBCs are most similar to the pathologist's annotations (2220).

### 4. Evaluation and Analysis

The evaluation of segmentation results on the test set mainly includes the classification evaluation of myeloblasts and other types of WBCs and the overall semantic segmentation evaluation of the segmentation model. Therefore, the following were calculated: the confusion matrix between the segmentation results and manual labels, the true positive (TP), true negative (TN), false positive (FP), and false negative (FN).

### 4.1. Evaluation Matrix

Pixel accuracy (PA), Intersection over Union (IoU), Precision, Recall, and Dice coefficient (DICE) were adopted to demonstrate the advantages of AMLcGAN. The class-wise evaluation of myeloblasts and other types of WBCs can explain the model's ability to capture local features. PA determines the ratio of the number of correctly identified pixels in the semantic segmentation result to the total number of pixels. PA is defined in formula (7):

$$PA = \frac{TP + TN}{TP + TN + FP + FN} \qquad ( 7 )$$

IoU quantifies the overlap ratio between model predictions and target ground truth. IoU is defined in formula (8):

$$IoU = \frac{TP}{TP + FP + FN} \qquad ( 8 )$$

Precision and Recall, respectively, calculate the ratio of the number of correctly identified pixels in the model prediction to the number of positive pixels predicted by the model and the number of target ground truth positive pixels. Precision and Recall are defined, respectively, in formulas (10) and (11):

$$\begin{matrix}Precision = \frac{TP}{TP + FP} & ( 9 ) \\ Recall = \frac{TP}{TP + FN} & ( 10 )\end{matrix}$$

DICE is a commonly used evaluation matrix in semantic segmentation, which measures the similarity between model predictions and target. DICE is defined in equation (11):

$$DICE = \frac{2 \times TP}{2 \times TP + FP + FN} \qquad ( 11 )$$

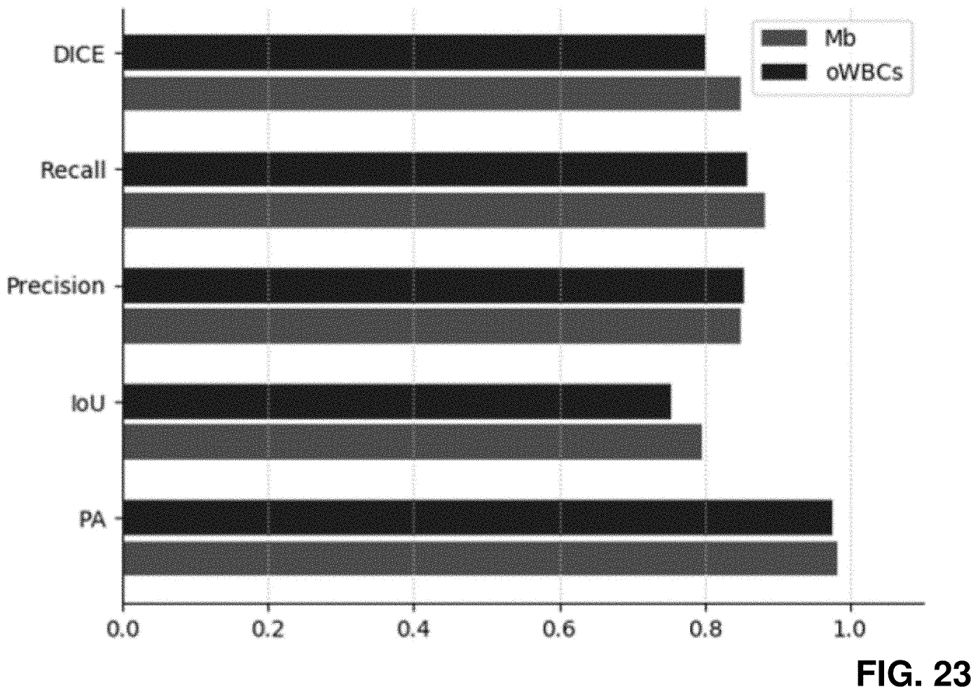

Referring to FIG. 23, illustrated is a graph showing the evaluation results of AMLcGAN on the test set, in connection with various aspects discussed herein.

As shown in FIG. 23, adversarial feature learning makes AMLcGAN superior in segmentation of myeloblasts and other types of white blood cells. As a baseline, AMLcGAN on average achieves 97.94% PA, 77.52% IoU, 85.13% Precision, 87.07% Recall and 82.51% DICE.

### 4.2. Comprehensive Analysis

Finally, three other popular segmentation networks were compared to show the advantages of AMLcGAN. The first is Unet, proposed for biomedical image segmentation, which uses a series of cascaded convolutional layers and a maximum pooling layer for down-sampling, a series of deconvolutional layers for up-sampling, and a series of skip connections between the feature maps of the encoder and decoder of the same scale to maintain the details of deep features. The second is SegNet, which uses a series of skip connections similar to Unet, but the decoder uses a series of cascaded nonlinear up-sampling and convolutional layers to up-sample the feature maps and reduce the time cost of model running. The third is the aforementioned Pix2pix. The four segmentation models used the same training epoch and training batch, and the same training set and test set. The class-wise comparative evaluation of different models on the test set is shown in Table 4 and Table 5.

TABLE 4
Myeloblast comparative evaluation

| Method | PA | IoU | Precision | Recall | DICE |
|---|---|---|---|---|---|
| Unet | 0.9706 | 0.7266 | 0.7771 | 0.8513 | 0.7933 |
| Segnet | 0.9701 | 0.7051 | 0.7752 | 0.8116 | 0.7719 |
| Pix2pix | 0.9668 | 0.7401 | 0.7748 | 0.9045 | 0.8141 |
| AMLcGAN | 0.9827 | 0.7965 | 0.8496 | 0.8831 | 0.8491 |

TABLE 5
Other types of white blood cells comparative evaluation

| Method | PA | IoU | Precision | Recall | DICE |
|---|---|---|---|---|---|
| Unet | 0.9719 | 0.7018 | 0.8001 | 0.8582 | 0.7546 |
| Segnet | 0.9701 | 0.7386 | 0.8097 | 0.8842 | 0.7921 |
| Pix2pix | 0.9709 | 0.7273 | 0.8185 | 0.8236 | 0.7764 |
| AMLcGAN | 0.9761 | 0.7539 | 0.8529 | 0.8583 | 0.8011 |

Table 4 and Table 5 show that AMLcGAN had the best performance in the class-wise evaluation of segmentation results, in which the average Recall index of the multi-category evaluation could reach the best 87.07% baseline among all comparison models. Unet, segnet and Pix2pix achieved average Recall indicators of 85.48%, 84.79%, and 86.41% respectively.

Finally, the third example study compared the PA and DICE averages of the evaluation results of each category as the global evaluation index of the segmentation models. The comparison results are shown in Table 6.

TABLE 6
Global comparative evaluation

| Method | Mean PA | Mean DICE |
|---|---|---|
| Unet | 0.9712 | 0.7739 |
| Segnet | 0.9701 | 0.7819 |
| Pix2pix | 0.9688 | 0.7951 |
| AMLcGAN | 0.9794 | 0.8249 |

The comparison results of Table 4, Table 5 and Table 6 show the great advantages of AMLcGAN in the segmentation of myeloblast. Compared with the existing segmentation networks shown in Table 6, the mean DICE of AMLcGAN significantly outperformed the Unet, Segnet and Pix2pix by 5.1%, 4.3%, and 2.98% respectively. Therefore, AMLcGAN was superior in prediction performance and applicability in the field of computer-aided diagnosis of blood diseases.

### 4.3. Comparing Segmented Blasts Between Target Groups

As it was declared earlier, one of the most important applications of myeloblasts segmentation is supporting clinicians in the prediction of whether an AML patient is going to relapse post-transplant. Since the blast quantity is associated with AML aggressiveness, the current gold standard for timely detection of relapse after bone marrow transplant is manual review of bone marrow aspirate and marrow to assess blast accumulation.

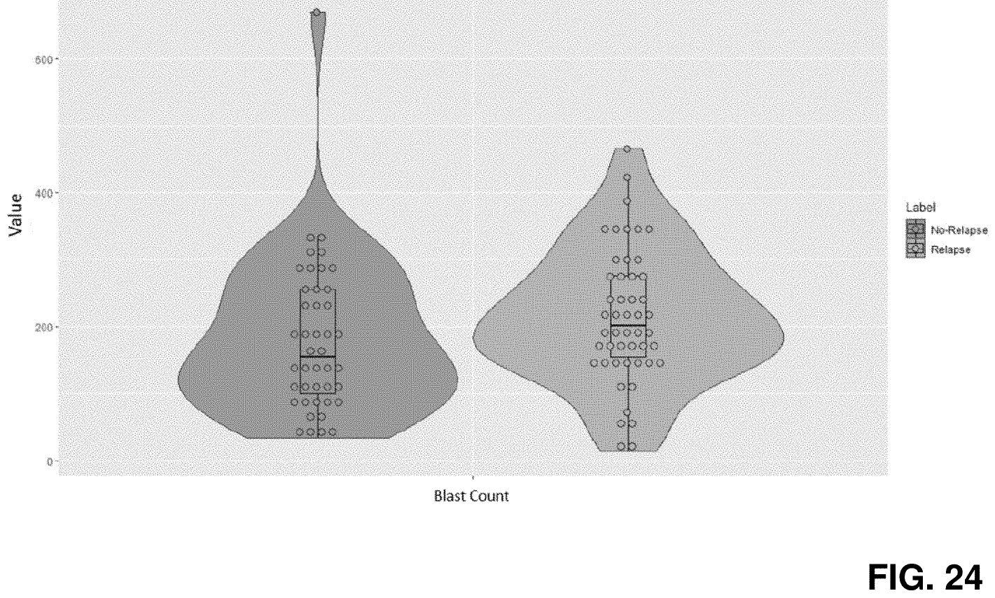

During this process a hematopathologist usually counts at least 200 random cells from a bone marrow aspirate specimen and determines if five percent or more of the cells are blasts, which would indicate relapse. In the third example study, a deep learning model for myeloblast automatic segmentation called AMLcGAN was developed, which can be used for the purpose of identifying AML patients who relapse after transplantation. Therefore, in order to further evaluate AMLcGAN on real data, the third example study collected a dataset of n=92 AML patients from UHCMC who underwent a bone marrow transplantation. For each patient, a Wright-Giemsa stained aspirate image is obtained six to eight weeks after transplantation. Of these patients n=44 experienced relapse. To compare the two groups of AML patients post-transplant, six non-overlapping 2048×2048 pixel tiles were first randomly selected from each aspirate image (overall 552 tiles from n=92 patients). AMLcGAN was then applied, the outputs were generated on these tiles, and the myeloblasts on every tile were counted. Referring to FIG. 24, illustrated are violin plots demonstrating the difference of blast counts obtained based on AMLcGAN between AML patients groups post-HCT, in connection with various aspects discussed herein. The violin plots in FIG. 24 show the differences in distribution of blast across relapse and no-relapse groups. According to FIG. 24, blasts counts in the relapse group are significantly higher than the no-relapse group (p-value=0.05), which is compatible with the expectation that a greater number of blasts corresponds with a more aggressive AML.

### 5. Conclusion and Discussion

The AMLcGAN model of the third example study, based on conditional adversarial feature learning, achieved automatic end-to-end semantic segmentation of multiple types of WBCs in Wright-Giemsa stained aspirate images, which can be helpful for clinical diagnosis, prognosis and prediction of blood diseases. AMLcGAN combines the advantages of deep feature learning and generative adversarial training. It not only perfectly achieved the semantic segmentation of white blood cells, but also distinguished myeloblasts from other types of WBCs at the feature level. When validated on 204 256×256 pathological images, AMLcGAN achieved accurate segmentation of WBCs.

Specifically, the global average pixel accuracy and average DICE of AMLcGAN reached 97.94% and 82.49%, respectively, and the PA on the two types of white blood cells reached 79.65% and 75.39%, respectively. This shows that the segmentation system can provide a very visual presentation for clinical application, which can assist clinicians in improving diagnosis efficiency.

The limitations of the third example study are mainly in two aspects: (1) This task only achieves the semantic segmentation of WBCs but did not address the segmentation of overlapping or adhering white blood cells. Various embodiments, however, can cascade a new post-processing network based on the current semantic segmentation result to achieve instance segmentation. (2) The method in the third example study can be used to verify the effectiveness and breadth of its application to data sets of other diseases. Various embodiments can employ AMLcGAN or a similar deep learning network based on techniques discussed herein to other diseases than those analyzed in the third example study.

### ADDITIONAL EMBODIMENTS

In various example embodiments, method(s) discussed herein can be implemented as computer executable instructions. Thus, in various embodiments, a computer-readable storage device can store computer executable instructions that, when executed by a machine (e.g., computer, processor), cause the machine to perform methods or operations described or claimed herein including operation(s) described in connection with methods 100, 200, or any other methods or operations described herein. While executable instructions associated with the listed methods are described as being stored on a computer-readable storage device, it is to be appreciated that executable instructions associated with other example methods or operations described or claimed herein can also be stored on a computer-readable storage device. In different embodiments, the example methods or operations described herein can be triggered in different ways. In one embodiment, a method or operation can be triggered manually by a user. In another example, a method or operation can be triggered automatically.

Embodiments discussed herein relate to constructing, training, and/or employing models that can automatically segment myeloblasts and/or determine a risk of relapse post-transplant for AML based on features of digitized WSI(s) of bone marrow aspirate that are not perceivable by the human eye, and involve computation that cannot be practically performed in the human mind. As one example, deep learning and/or machine learning models as described herein cannot be implemented in the human mind or with pencil and paper. Embodiments thus perform actions, steps, processes, or other actions that are not practically performed in the human mind, at least because they require a processor or circuitry to access digitized WSIs stored in a computer memory and to extract or compute features that are based on the digitized WSIs and not on properties of tissue or the images that are perceivable by the human eye. Embodiments described herein can use a combined order of specific rules, elements, operations, or components that render information into a specific format that can then be used and applied to create desired results more accurately, more consistently, and with greater reliability than existing approaches, thereby producing the technical effect of improving the performance of the machine, computer, or system with which embodiments are implemented.

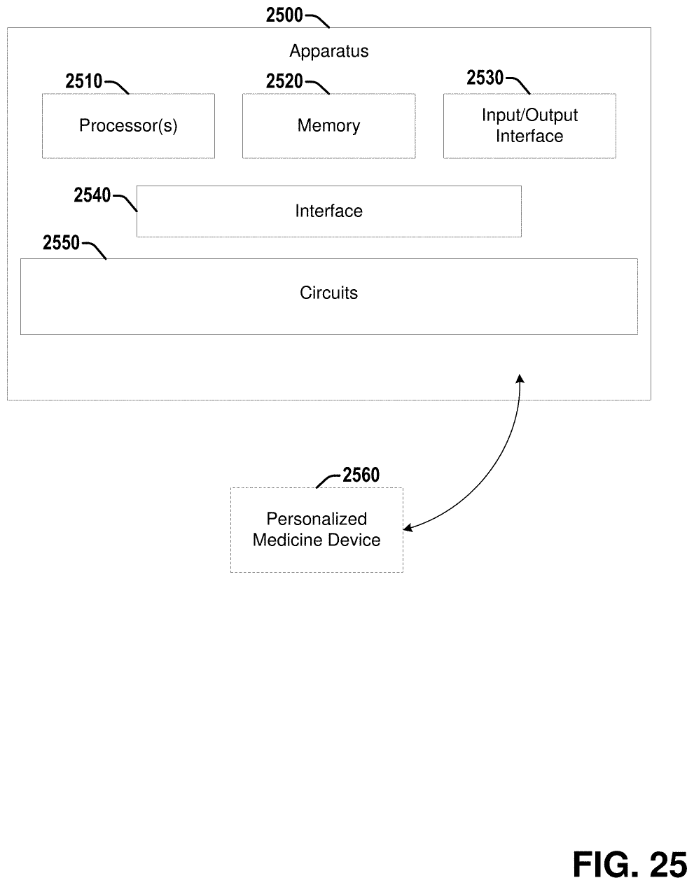

Referring to FIG. 25, illustrated is a diagram of an example apparatus 2500 that can facilitate constructing, training, and/or employing models that can automatically segment myeloblasts and/or determine a risk of relapse post-transplant for AML based on features of digitized WSI(s) of bone marrow aspirate, in connection with various aspects discussed herein. Apparatus 2500 can be configured to perform various techniques discussed herein, for example, various operations discussed in connection with sets of operations 100, 200, and/or other methods described herein. Apparatus 2500 can comprise one or more processors 2510 and memory 2520. Processor(s) 2510 can, in various embodiments, comprise circuitry such as, but not limited to, one or more single-core or multi-core processors. Processor(s) 2510 can include any combination of general-purpose processors and dedicated processors (e.g., graphics processors, application processors, etc.). The processor(s) can be coupled with and/or can comprise memory (e.g., of memory 2520) or storage and can be configured to execute instructions stored in the memory 2520 or storage to enable various apparatus, applications, or operating systems to perform operations and/or methods discussed herein. Memory 2520 can be configured to store one or more digital whole slide images (WSIs) (e.g., obtained via optical microscopes, etc.) of bone marrow aspirate. Each of the digital WSI(s) can comprise a plurality of pixels or voxels, each pixel or voxel having an associated intensity. Memory 2520 can be further configured to store additional data involved in performing operations discussed herein, such as for constructing, training, and/or employing models that can automatically segment myeloblasts and/or determine a risk of relapse post-transplant for AML based on features of digitized WSI(s) of bone marrow aspirate, in connection with various aspects discussed herein.

Apparatus 2500 can also comprise an input/output (I/O) interface 2530 (e.g., associated with one or more I/O devices), a set of circuits 2550, and an interface 2540 that connects the processor(s) 2510, the memory 2520, the I/O interface 2530, and the set of circuits 2550. I/O interface 2530 can be configured to transfer data between memory 2520, processor 2510, circuits 2550, and external devices, for example, medical imaging device(s) (e.g., optical microscopy system(s), etc.), and/or one or more remote devices for receiving inputs and/or providing outputs to a clinician, patient, etc., such as optional personalized medicine device 2560.

The processor(s) 2510 and/or one or more circuits of the set of circuits 2550 can perform one or more acts associated with a method or set of operations discussed herein, such as set of operations 100, 200, etc. In various embodiments, different acts (e.g., different operations of a set of operations) can be performed by the same or different processor(s) 2510 and/or one or more circuits of the set of circuits 2550.

Apparatus 2500 can optionally further comprise personalized medicine device 2560. Apparatus 2500 can be configured to provide the segmentation results, risk of relapse, and/or other data to personalized medicine device 2560. Personalized medicine device 2560 may be, for example, a computer assisted diagnosis (CADx) system or other type of personalized medicine device that can be used to facilitate monitoring and/or treatment of an associated medical condition. In some embodiments, processor(s) 2510 and/or one or more circuits of the set of circuits 2550 can be further configured to control personalized medicine device 2560 to display the segmented myeloblasts, values of one or more size/shape/texture feature(s), risk of relapse, and/or other data on a computer monitor, a smartphone display, a tablet display, or other displays.

Examples herein can include subject matter such as an apparatus, an optical microscopy system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system for performing any operations and/or methods discussed herein, according to embodiments and examples described.

Various embodiments can facilitate automated analysis methods, operations, systems, apparatus, or other embodiments, for predicting relapse in AML, facilitating identifying patients who may benefit from additional therapy following transplant.

One embodiment includes a non-transitory computer-readable storage device storing computer-executable instructions that when executed control a processor to perform operations for predicting likelihood of relapse in AML, the operations comprising: accessing a digitized stained post-transplant aspirate smear associated with a patient demonstrating AML; segmenting at least one myeloblast represented in the digitized Wright-Giemsa stained post-transplant aspirate smear using an automated blast segmentation model; extracting a set of discriminative features from the at least one myeloblast; providing the set of discriminative features to a machine learning model configured to generate a prediction of AML relapse; receiving, from the machine learning model, a prediction of AML relapse; and generating a classification of the patient as likely to experience AML relapse, or unlikely to experience AML relapse based, at least in part, on the prediction of AML relapse. Embodiments may further display the classification. Embodiments may further display the prediction, the set of discriminative features, the digitized Wright-Giemsa stained post-transplant aspirate smear, an operating parameter of the automated blast segmentation model, or an operating parameter of the machine learning model configured to generate a prediction of AML relapse. In one embodiment, the digitized stained post-transplant aspirate smear associated with a patient demonstrating AML comprises a digitized Wright-Giemsa stained post-transplant aspirate smear associated with a patient demonstrating AML. The digitized stained post-transplant aspirate smear comprises a plurality of pixels, a pixel having an associated intensity.

In one embodiment the automated blast segmentation model comprises a conditional generative adversarial network (cGAN).

In one embodiment, the set of discriminative features comprises at least one Haralick feature, and at least one Fractal dimension feature.

Embodiments may further comprise training an automated blast segmentation model to distinguish blasts on Wright-Giemsa stained post-transplant aspirate smear imagery according to various techniques described herein.

Embodiments may further comprise training a machine learning model configured to generate a prediction of AML relapse based on discriminative features extracted from a digitized Wright-Giemsa stained post-transplant aspirate smear. In one embodiment, the machine learning model is a linear discriminant analysis (LDA) classifier.

Operations, methods, and other embodiments described herein include acquiring electronic data, reading from a computer file, receiving a computer file, reading from a computer memory, or other computerized activity not practically performed in a human mind, at least because a conditional generative adversarial network (cGAN) cannot be practically performed or implemented in a human mind. For example, accessing a digitized Wright-Giemsa stained post-transplant aspirate smear, or segmenting at least one myeloblast represented in the digitized Wright-Giemsa stained post-transplant aspirate smear using an automated blast segmentation model, include acquiring electronic data, reading from a computer file, receiving a computer file, reading from a computer memory, or other computerized activity not practically performed in a human mind.

Example 1 is a non-transitory computer-readable medium storing computer-executable instructions that, when executed, cause a processor to perform operations, comprising: accessing a digital whole slide image (WSI) comprising a post-transplant bone marrow aspirate from a patient that has acute myeloid leukemia (AML); segmenting one or more myeloblasts on the digital WSI; extracting one or more features from the segmented one or more myeloblasts; providing the one or more features extracted from the segmented one or more myeloblasts to a trained machine learning model; and receiving, from the trained machine learning model, an indication of a risk of relapse of the AML.

Example 2 comprises the subject matter of any variation(s) of any of example(s) 1, wherein the machine learning model is one of, or an ensemble of two or more of, a logistic regression model, a Cox regression model, a Least Absolute Shrinkage and Selection Operator (LASSO) regression model, a naïve Bayes classifier, a support vector machine (SVM) with a linear kernel, a SVM with a radial basis function (RBF) kernel, a linear discriminant analysis (LDA) classifier, a quadratic discriminant analysis (QDA) classifier, a logistic regression classifier, a decision tree, a random forest, a diagonal LDA, a diagonal QDA, a neural network, an AdaBoost algorithm, an elastic net, a Gaussian process classification, or a nearest neighbors classification.

Example 3 comprises the subject matter of any variation(s) of any of example(s) 1-2, wherein the one or more features comprise at least one of: at least one Haralick feature of the segmented one or more myeloblasts, a statistic of the at least one Haralick feature, at least one fractal dimension (FD) feature of the segmented one or more myeloblasts, or the statistic of the at least one FD feature.

Example 4 comprises the subject matter of any variation(s) of any of example(s) 3, wherein the statistic is one of a mean, a median, a standard deviation, a skewness, a kurtosis, a range, a minimum, a maximum, a percentile, or histogram frequencies.

Example 5 comprises the subject matter of any variation(s) of any of example(s) 3-4, wherein the at least one Haralick feature of the segmented one or more myeloblasts comprises one or more of an intensity entropy, an information measure, or a contrast inverse moment.

Example 6 comprises the subject matter of any variation(s) of any of example(s) 3-5, wherein the at least one FD feature of the segmented one or more myeloblasts comprises one or more of an entropy in a time series or a two-dimensional (2D) FD of myeloblast texture.

Example 7 comprises the subject matter of any variation(s) of any of example(s) 1-6, wherein segmenting the one or more myeloblasts on the digital WSI comprises segmenting the one or more myeloblasts on the digital WSI via a deep learning (DL) model.

Example 8 comprises the subject matter of any variation(s) of any of example(s) 7, wherein the DL model is a conditional generative adversarial networks (cGAN) or is based on the cGAN.

Example 9 comprises the subject matter of any variation(s) of any of example(s) 8, wherein the DL model employs an optimization objective based at least on a conditional adversarial loss.

Example 10 comprises the subject matter of any variation(s) of any of example(s) 8-9, wherein the DL model employs an optimization objective based at least on a feature matching loss.

Example 11 is a non-transitory computer-readable medium storing computer-executable instructions that, when executed, cause a processor to perform operations, comprising: accessing a training set comprising a plurality of digital whole slide images (WSIs), wherein each digital WSI of the plurality of digital WSIs comprises an associated post-transplant bone marrow aspirate from an associated patient that has acute myeloid leukemia (AML), wherein each digital WSI of the plurality of digital WSIs has a known associated outcome for the associated patient of that digital WSI, wherein the known associated outcome is one of a relapse or a non-relapse; for each digital WSI of the training set: segmenting one or more associated myeloblasts on that digital WSI; and extracting an associated value for each of a plurality of features from the associated segmented cancer nuclei of that digital WSI; determining a set of best features from the plurality of features, based at least in part on the known associated outcome for each digital WSI of the training set and on the associated values for each of the plurality of features for each digital WSI of the training set; and constructing a machine learning model configured to determine an additional associated outcome for an additional digital WSI based at least in part on the set of best features.

Example 12 comprises the subject matter of any variation(s) of any of example(s) 11, wherein determining the set of best features comprises determining the set of best features via a least absolute shrinkage and selection operator (LASSO).

Example 13 comprises the subject matter of any variation(s) of any of example(s) 11-12, wherein the machine learning model is one of, or an ensemble of two or more of, a logistic regression model, a Cox regression model, a Least Absolute Shrinkage and Selection Operator (LASSO) regression model, a naïve Bayes classifier, a support vector machine (SVM) with a linear kernel, a SVM with a radial basis function (RBF) kernel, a linear discriminant analysis (LDA) classifier, a quadratic discriminant analysis (QDA) classifier, a logistic regression classifier, a decision tree, a random forest, a diagonal LDA, a diagonal QDA, a neural network, an AdaBoost algorithm, an elastic net, a Gaussian process classification, or a nearest neighbors classification.

Example 14 comprises the subject matter of any variation(s) of any of example(s) 11-13, wherein the plurality of features comprise at least one of: at least one Haralick feature of the segmented one or more myeloblasts, a statistic of the at least one Haralick feature, at least one fractal dimension (FD) feature of the segmented one or more myeloblasts, or the statistic of the at least one FD feature.

Example 15 comprises the subject matter of any variation(s) of any of example(s) 14, wherein the statistic is one of a mean, a median, a standard deviation, a skewness, a kurtosis, a range, a minimum, a maximum, a percentile, or histogram frequencies.

Example 16 comprises the subject matter of any variation(s) of any of example(s) 14-15, wherein the at least one Haralick feature of the segmented one or more myeloblasts comprises one or more of an intensity entropy, an information measure, or a contrast inverse moment.

Example 17 comprises the subject matter of any variation(s) of any of example(s) 14-16, wherein the at least one FD feature of the segmented one or more myeloblasts comprises one or more of an entropy in a time series or a two-dimensional (2D) FD of myeloblast texture.

Example 18 comprises the subject matter of any variation(s) of any of example(s) 14-17, wherein, for each digital WSI of the training set, segmenting the one or more associated myeloblasts on that digital WSI comprises segmenting the one or more myeloblasts on that digital WSI via a deep learning (DL) model.

Example 19 comprises the subject matter of any variation(s) of any of example(s) 18, wherein the DL model is a conditional generative adversarial networks (cGAN) or is based on the cGAN.

Example 20 comprises the subject matter of any variation(s) of any of example(s) 19, wherein the DL model employs an optimization objective based on at least one of a conditional adversarial loss or a feature matching loss.

Example 21 is an apparatus, comprising: memory configured to store at least a portion of a digital whole slide image (WSI) comprising a post-transplant bone marrow aspirate from a patient that has acute myeloid leukemia (AML); one or more processors configured to perform operations comprising: segmenting one or more myeloblasts on the digital WSI; extracting one or more features from the segmented one or more myeloblasts; providing the one or more features extracted from the segmented one or more myeloblasts to a trained machine learning model; and receiving, from the trained machine learning model, an indication of a risk of relapse of the AML.

Example 22 comprises the subject matter of any variation(s) of any of example(s) 21, wherein the machine learning model is one of, or an ensemble of two or more of, a logistic regression model, a Cox regression model, a Least Absolute Shrinkage and Selection Operator (LASSO) regression model, a naïve Bayes classifier, a support vector machine (SVM) with a linear kernel, a SVM with a radial basis function (RBF) kernel, a linear discriminant analysis (LDA) classifier, a quadratic discriminant analysis (QDA) classifier, a logistic regression classifier, a decision tree, a random forest, a diagonal LDA, a diagonal QDA, a neural network, an AdaBoost algorithm, an elastic net, a Gaussian process classification, or a nearest neighbors classification.

Example 23 comprises the subject matter of any variation(s) of any of example(s) 21-22, wherein the one or more features comprise at least one of: at least one Haralick feature of the segmented one or more myeloblasts, a statistic of the at least one Haralick feature, at least one fractal dimension (FD) feature of the segmented one or more myeloblasts, or the statistic of the at least one FD feature.

Example 24 comprises the subject matter of any variation(s) of any of example(s) 23, wherein the statistic is one of a mean, a median, a standard deviation, a skewness, a kurtosis, a range, a minimum, a maximum, a percentile, or histogram frequencies.

Example 25 comprises the subject matter of any variation(s) of any of example(s) 23-24, wherein the at least one Haralick feature of the segmented one or more myeloblasts comprises one or more of an intensity entropy, an information measure, or a contrast inverse moment.

Example 26 comprises the subject matter of any variation(s) of any of example(s) 23-25, wherein the at least one FD feature of the segmented one or more myeloblasts comprises one or more of an entropy in a time series or a two-dimensional (2D) FD of myeloblast texture.

Example 27 comprises the subject matter of any variation(s) of any of example(s) 21-26, wherein segmenting the one or more myeloblasts on the digital WSI comprises segmenting the one or more myeloblasts on the digital WSI via a deep learning (DL) model.

Example 28 comprises an apparatus comprising means for executing any of the described operations of examples 1-27.

Example 29 comprises a computer-readable medium that stores instructions for execution by a processor to perform any of the described operations of examples 1-27.

Example 30 comprises an apparatus comprising: a memory; and one or more processors configured to: perform any of the described operations of examples 1-27.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A non-transitory computer-readable medium storing computer-executable instructions that, when executed, cause a processor to perform operations, comprising:
accessing a digital whole slide image (WSI) comprising a post-transplant bone marrow aspirate from a patient that has acute myeloid leukemia (AML);
segmenting one or more myeloblasts on the digital WSI;
extracting one or more features from the segmented one or more myeloblasts;
providing the one or more features extracted from the segmented one or more myeloblasts to a trained machine learning model; and
receiving, from the trained machine learning model, an indication of a risk of relapse of the AML.

2. The non-transitory computer-readable medium of claim 1, wherein the machine learning model is one of, or an ensemble of two or more of, a logistic regression model, a Cox regression model, a Least Absolute Shrinkage and Selection Operator (LASSO) regression model, a naïve Bayes classifier, a support vector machine (SVM) with a linear kernel, a SVM with a radial basis function (RBF) kernel, a linear discriminant analysis (LDA) classifier, a quadratic discriminant analysis (QDA) classifier, a logistic regression classifier, a decision tree, a random forest, a diagonal LDA, a diagonal QDA, a neural network, an AdaBoost algorithm, an elastic net, a Gaussian process classification, or a nearest neighbors classification.

3. The non-transitory computer-readable medium of claim 1, wherein the one or more features comprise at least one of: at least one Haralick feature of the segmented one or more myeloblasts, a statistic of the at least one Haralick feature, at least one fractal dimension (FD) feature of the segmented one or more myeloblasts, or the statistic of the at least one FD feature.

4. The non-transitory computer-readable medium of claim 3, wherein the statistic is one of a mean, a median, a standard deviation, a skewness, a kurtosis, a range, a minimum, a maximum, a percentile, or histogram frequencies.

5. The non-transitory computer-readable medium of claim 3, wherein the at least one Haralick feature of the segmented one or more myeloblasts comprises one or more of an intensity entropy, an information measure, or a contrast inverse moment.

6. The non-transitory computer-readable medium of claim 3, wherein the at least one FD feature of the segmented one or more myeloblasts comprises one or more of an entropy in a time series or a two-dimensional (2D) FD of myeloblast texture.

7. The non-transitory computer-readable medium of claim 1, wherein segmenting the one or more myeloblasts on the digital WSI comprises segmenting the one or more myeloblasts on the digital WSI via a deep learning (DL) model.

8. The non-transitory computer-readable medium of claim 7, wherein the DL model is a conditional generative adversarial networks (cGAN) or is based on the cGAN.

9. The non-transitory computer-readable medium of claim 8, wherein the DL model employs an optimization objective based at least on a conditional adversarial loss.

10. The non-transitory computer-readable medium of claim 8, wherein the DL model employs an optimization objective based at least on a feature matching loss.

11. A non-transitory computer-readable medium storing computer-executable instructions that, when executed, cause a processor to perform operations, comprising:
accessing a training set comprising a plurality of digital whole slide images (WSIs), wherein each digital WSI of the plurality of digital WSIs comprises an associated post-transplant bone marrow aspirate from an associated patient that has acute myeloid leukemia (AML), wherein each digital WSI of the plurality of digital WSIs has a known associated outcome for the associated patient of that digital WSI, wherein the known associated outcome is one of a relapse or a non-relapse;
for each digital WSI of the training set:
segmenting one or more associated myeloblasts on that digital WSI; and
extracting an associated value for each of a plurality of features from the associated segmented cancer nuclei of that digital WSI;
determining a set of best features from the plurality of features, based at least in part on the known associated outcome for each digital WSI of the training set and on the associated values for each of the plurality of features for each digital WSI of the training set; and
constructing a machine learning model configured to determine an additional associated outcome for an additional digital WSI based at least in part on the set of best features.

12. The non-transitory computer-readable medium of claim 11, wherein determining the set of best features comprises determining the set of best features via a least absolute shrinkage and selection operator (LASSO).

13. The non-transitory computer-readable medium of claim 11, wherein the machine learning model is one of, or an ensemble of two or more of, a logistic regression model, a Cox regression model, a Least Absolute Shrinkage and Selection Operator (LASSO) regression model, a naïve Bayes classifier, a support vector machine (SVM) with a linear kernel, a SVM with a radial basis function (RBF) kernel, a linear discriminant analysis (LDA) classifier, a quadratic discriminant analysis (QDA) classifier, a logistic regression classifier, a decision tree, a random forest, a diagonal LDA, a diagonal QDA, a neural network, an AdaBoost algorithm, an elastic net, a Gaussian process classification, or a nearest neighbors classification.

14. The non-transitory computer-readable medium of claim 11, wherein the plurality of features comprise at least one of: at least one Haralick feature of the segmented one or more myeloblasts, a statistic of the at least one Haralick feature, at least one fractal dimension (FD) feature of the segmented one or more myeloblasts, or the statistic of the at least one FD feature.

15. The non-transitory computer-readable medium of claim 14, wherein the statistic is one of a mean, a median, a standard deviation, a skewness, a kurtosis, a range, a minimum, a maximum, a percentile, or histogram frequencies.

16. The non-transitory computer-readable medium of claim 14, wherein the at least one Haralick feature of the segmented one or more myeloblasts comprises one or more of an intensity entropy, an information measure, or a contrast inverse moment.

17. The non-transitory computer-readable medium of claim 14, wherein the at least one FD feature of the segmented one or more myeloblasts comprises one or more of an entropy in a time series or a two-dimensional (2D) FD of myeloblast texture.

18. The non-transitory computer-readable medium of claim 14, wherein, for each digital WSI of the training set, segmenting the one or more associated myeloblasts on that digital WSI comprises segmenting the one or more myeloblasts on that digital WSI via a deep learning (DL) model.

19. The non-transitory computer-readable medium of claim 18, wherein the DL model is a conditional generative adversarial networks (cGAN) or is based on the cGAN.

20. The non-transitory computer-readable medium of claim 19, wherein the DL model employs an optimization objective based on at least one of a conditional adversarial loss or a feature matching loss.

21. An apparatus, comprising:
memory configured to store at least a portion of a digital whole slide image (WSI) comprising a post-transplant bone marrow aspirate from a patient that has acute myeloid leukemia (AML);
one or more processors configured to perform operations comprising:
segmenting one or more myeloblasts on the digital WSI;
extracting one or more features from the segmented one or more myeloblasts;
providing the one or more features extracted from the segmented one or more myeloblasts to a trained machine learning model; and
receiving, from the trained machine learning model, an indication of a risk of relapse of the AML.

22. The apparatus of claim 21, wherein the machine learning model is one of, or an ensemble of two or more of, a logistic regression model, a Cox regression model, a Least Absolute Shrinkage and Selection Operator (LASSO) regression model, a naïve Bayes classifier, a support vector machine (SVM) with a linear kernel, a SVM with a radial basis function (RBF) kernel, a linear discriminant analysis (LDA) classifier, a quadratic discriminant analysis (QDA) classifier, a logistic regression classifier, a decision tree, a random forest, a diagonal LDA, a diagonal QDA, a neural network, an AdaBoost algorithm, an elastic net, a Gaussian process classification, or a nearest neighbors classification.

23. The apparatus of claim 21, wherein the one or more features comprise at least one of: at least one Haralick feature of the segmented one or more myeloblasts, a statistic of the at least one Haralick feature, at least one fractal dimension (FD) feature of the segmented one or more myeloblasts, or the statistic of the at least one FD feature.

24. The apparatus of claim 23, wherein the statistic is one of a mean, a median, a standard deviation, a skewness, a kurtosis, a range, a minimum, a maximum, a percentile, or histogram frequencies.

25. The apparatus of claim 23, wherein the at least one Haralick feature of the segmented one or more myeloblasts comprises one or more of an intensity entropy, an information measure, or a contrast inverse moment.

26. The apparatus of claim 23, wherein the at least one FD feature of the segmented one or more myeloblasts comprises one or more of an entropy in a time series or a two-dimensional (2D) FD of myeloblast texture.

27. The apparatus of claim 21, wherein segmenting the one or more myeloblasts on the digital WSI comprises segmenting the one or more myeloblasts on the digital WSI via a deep learning (DL) model.
